SEQUENCE LISTING (52 sequences)

SEQ ID NO: 1 - protein, 447 aa, synthetic construct
QVQLQQSGPELVKPETSVKISCKASGYTFTSYYIHWVKQRPGQGLEWIGWIYPGNVNTKYNERFKGKATLTADKSSNTAHMQLTSLTSEDSAVYFCARSNPYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 117 aa, synthetic construct
QVQLQQSGPELKKPEASVKISCKASGYTFTSYYIHWVKQAPGQGLEWIGWIYPGNVNTKYNERFKGRATLTADKSTNTAHMELSSLTSEDSAVYFCARSNPYVMDYWGQGTSVTVSS

SEQ ID NO: 3 - protein, 117 aa, synthetic construct
QVQLVQSGSELKKPGASVKISCKASGYTFTSYYIHWVKQAPGQGLEWIGWIYPGNVNTKYNERFKGRATLTADKSTNTAYMELSSLRSEDTAVYFCARSNPYVMDYWGQGTSVTVSS

SEQ ID NO: 4 - protein, 117 aa, synthetic construct
QVQLVQSGSELKKPGASVKVSCKASGYTFTSYYIHWVRQAPGQGLEWIGWIYPGNVNTKYNERFKGRATLTADKSTNTAYMELSSLRSEDTAVYFCARSNPYVMDYWGQGTSVTVSS

SEQ ID NO: 5 - protein, 117 aa, synthetic construct
QVQLVQSGSELKKPGASVKVSCKASGYTFTSYYIHWVRQAPGQGLEWIGWIYPGNVNTKYNERFKGRVTITADKSTNTAYMELSSLRSEDTAVYYCARSNPYVMDYWGQGTSVTVSS

SEQ ID NO: 6 - protein, 117 aa, synthetic construct
QVQLVQSGSELKKPGASVKVSCKASGYTFTSYYIHWVRQAPGQGLEWMGWIYPGNVNTKYNERFQGRVTITADKSTSTAYMELSSLRSEDTAVYYCARSNPYVMDYWGQGTSVTVSS

SEQ ID NO: 7 - protein, 214 aa, synthetic construct
SIVMTQTPKFLLVSAGDRVTITCKASQSVNNDVAWYQQKPGLSPELLMYYASNRFTGVPDRFTGSGYGTDFTFTISSVQAEDLAIYFCQQAYRSPYTFGGGTKLEIQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 107 aa, synthetic construct
SIVMTQSPSFLLASAGDRVTITCKASQSVNNDVAWYQQKPGLAPELLMYYASNRFTGVPDRFTGSGYGTDFTLTISSLQAEDLAIYFCQQAYRSPYTFGQGTKLEIK

SEQ ID NO: 9 - protein, 107 aa, synthetic construct
SIVMTQSPSTLSASVGDRVTITCKASQSVNNDVAWYQQKPGKAPKLLMYYASNRFTGVPDRFTGSGYGTDFTLTISSLQAEDLAIYFCQQAYRSPYTFGQGTKLEIK

SEQ ID NO: 10 - protein, 107 aa, synthetic construct
DIQMTQSPSTLSASVGDRVTITCKASQSVNNDVAWYQQKPGKAPKLLMYYASNRFTGVPDRFSGSGYGTDFTLTISSLQAEDLAIYFCQQAYRSPYTFGQGTKLEIK

SEQ ID NO: 11 - protein, 107 aa, synthetic construct
DIQMTQSPSTLSASVGDRVTITCRASQSVNNDVAWYQQKPGKAPKLLMYYASNRFTGVPDRFSGSGYGTDFTLTISSLQAEDLAIYFCQQAYRSPYTFGQGTKLEIK

SEQ ID NO: 12 - protein, 107 aa, synthetic construct
DIQMTQSPSTLSASVGDRVTITCRASQSVNNDVAWYQQKPGKAPKLLMYYASNRFTGVPDRFSGSGYGTDFTLTISSLQAEDVAVYFCQQAYRSPYTFGQGTKLEIK

SEQ ID NO: 13 - protein, 117 aa, synthetic construct
  VARIANT 5: note = X = Q or V
  VARIANT 9: note = X=P or S
  VARIANT 15: note = X=E or G
  VARIANT 20: note = X=I or V
  VARIANT 38: note = X=K or R
  VARIANT 48: note = X=I or M
  VARIANT 65: note = X=K or Q
  VARIANT 68: note = X=A or V
  VARIANT 70: note = X=L or I
  VARIANT 77: note = X=N or S
  VARIANT 80: note = X=H or Y
  VARIANT 87: note = X=T or R
  VARIANT 91: note = X=S or T
  VARIANT 95: note = X=F or Y
QVQLXQSGXELKKPXASVKXSCKASGYTFTSYYIHWVXQAPGQGLEWXGWIYPGNVNTKYNERFXGRXTXTADKSTXTAXMELSSLXSEDXAVYXCARSNPYVMDYWGQGTSVTVSS

SEQ ID NO: 14 - protein, 107 aa, synthetic construct
  VARIANT 1: note = X=S or D
  VARIANT 3: note = X=V or Q
  VARIANT 10: note = X=F or T
  VARIANT 12: note = L or S
  VARIANT 15: note = X=A or V
  VARIANT 24: note = X=K or R
  VARIANT 42: note = X=L or K
  VARIANT 45: note = X=E or K
  VARIANT 63: note = X=T or S
  VARIANT 83: note = L or V
  VARIANT 85: note = X=I or V
XIXMTQSPSXLXASXGDRVTITCXASQSVNNDVAWYQQKPGXAPXLLMYYASNRFTGVPDRFXGSGYGTDFTLTISSLQAEDXAXYFCQQAYRSPYTFGQGTKLEIK

SEQ ID NO: 15 - protein, 5 aa, synthetic construct
SYYIH

SEQ ID NO: 16 - protein, 17 aa, synthetic construct
  VARIANT 16: note = X=K or Q
WIYPGNVNTKYNERFXG

SEQ ID NO: 17 - protein, 8 aa, synthetic construct
SNPYVMDY

SEQ ID NO: 18 - protein, 11 aa, synthetic construct
  VARIANT 1: note = X=K or R
XASQSVNNDVA

SEQ ID NO: 19 - protein, 7 aa, synthetic construct
YASNRFT

SEQ ID NO: 20 - protein, 9 aa, synthetic construct
QQAYRSPYT

SEQ ID NO: 21 - protein, 8 aa, synthetic construct
GYTFTSYY

SEQ ID NO: 22 - protein, 8 aa, synthetic construct
IYPGNVNT

SEQ ID NO: 23 - protein, 10 aa, synthetic construct
ARSNPYVMDY

SEQ ID NO: 24 - protein, 6 aa, synthetic construct
QSVNND

SEQ ID NO: 25-26 - skipped (2 entries)

SEQ ID NO: 27 - protein, 17 aa, synthetic construct
WIYPGNVNTKYNERFKG

SEQ ID NO: 28 - protein, 17 aa, synthetic construct
WIYPGNVNTKYNERFQG

SEQ ID NO: 29 - protein, 11 aa, synthetic construct
KASQSVNNDVA

SEQ ID NO: 30 - protein, 11 aa, synthetic construct
RASQSVNNDVA

SEQ ID NO: 31 - protein, 30 aa, synthetic construct
QVQLQQSGPELKKPEASVKISCKASGYTFT

SEQ ID NO: 32 - protein, 30 aa, synthetic construct
QVQLVQSGSELKKPGASVKISCKASGYTFT

SEQ ID NO: 33 - protein, 30 aa, synthetic construct
QVQLVQSGSELKKPGASVKVSCKASGYTFT

SEQ ID NO: 34 - protein, 14 aa, synthetic construct
WVKQAPGQGLEWIG

SEQ ID NO: 35 - protein, 14 aa, synthetic construct
WVRQAPGQGLEWIG

SEQ ID NO: 36 - protein, 14 aa, synthetic construct
WVRQAPGQGLEWMG

SEQ ID NO: 37 - protein, 32 aa, synthetic construct
RATLTADKSTNTAHMELSSLTSEDSAVYFCAR

SEQ ID NO: 38 - protein, 32 aa, synthetic construct
RATLTADKSTNTAYMELSSLRSEDTAVYFCAR

SEQ ID NO: 39 - protein, 32 aa, synthetic construct
RVTITADKSTNTAYMELSSLRSEDTAVYYCAR

SEQ ID NO: 40 - protein, 32 aa, synthetic construct
RVTITADKSTSTAYMELSSLRSEDTAVYYCAR

SEQ ID NO: 41 - protein, 11 aa, synthetic construct
WGQGTSVTVSS

SEQ ID NO: 42 - protein, 23 aa, synthetic construct
SIVMTQSPSFLLASAGDRVTITC

SEQ ID NO: 43 - protein, 23 aa, synthetic construct
SIVMTQSPSTLSASVGDRVTITC

SEQ ID NO: 44 - protein, 23 aa, synthetic construct
DIQMTQSPSTLSASVGDRVTITC

SEQ ID NO: 45 - protein, 15 aa, synthetic construct
WYQQKPGLAPELLMY

SEQ ID NO: 46 - protein, 15 aa, synthetic construct
WYQQKPGKAPKLLMY

SEQ ID NO: 47 - protein, 32 aa, synthetic construct
GVPDRFTGSGYGTDFTLTISSLQAEDLAIYFC

SEQ ID NO: 48 - protein, 32 aa, synthetic construct
GVPDRFSGSGYGTDFTLTISSLQAEDLAIYFC

SEQ ID NO: 49 - protein, 32 aa, synthetic construct
GVPDRFSGSGYGTDFTLTISSLQAEDVAVYFC

SEQ ID NO: 50 - protein, 10 aa, synthetic construct
FGQGTKLEIK

SEQ ID NO: 51 - protein, 447 aa, synthetic construct
QVQLVQSGSELKKPGASVKVSCKASGYTFTSYYIHWVRQAPGQGLEWIGWIYPGNVNTKYNERFKGRVTITADKSTNTAYMELSSLRSEDTAVYYCARSNPYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 214 aa, synthetic construct
DIQMTQSPSTLSASVGDRVTITCRASQSVNNDVAWYQQKPGKAPKLLMYYASNRFTGVPDRFSGSGYGTDFTLTISSLQAEDVAVYFCQQAYRSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0014269 A1
(43) Pub. Date: Jan. 15, 2026

(54) HUMANIZED ANTIBODIES AGAINST NECTIN-4 AND DRUG CONJUGATES THEREOF

(71) Applicant: NECTIN THERAPEUTICS LTD., Jerusalem (IL)

(72) Inventors: Pinchas TSUKERMAN, Jerusalem (IL); Anas ATIEH, Jerusalem (IL); Akram OBIEDAT, Jerusalem (IL); Alon VITENSHTEIN, Jerusalem (IL); Guy CINAMON, Tel Aviv (IL)

(73) Assignee: NECTIN THERAPEUTICS LTD., Jerusalem (IL)

(21) Appl. No.: 18/994,842

(22) PCT Filed: Sep. 19, 2023
(86) PCT No.: PCT/IL2023/051016

§ 371 Date: Jan. 15, 2025

Related U.S. Application Data
(60) Provisional application No. 63/408,106, filed on Sep. 20, 2022.

(51) Int. Cl. A61K 47/68; A61P 35/00; C07K 16/28

(52) U.S. Cl. CPC A61K 47/6851; A61K 47/68031; A61P 35/00; C07K 16/2803; C07K 2317/24; C07K 2317/33; C07K 2317/92

(57) ABSTRACT

The present invention provides humanized antibodies against Nectin-4 and antibody drug conjugates (ADCs) of the humanized antibodies and use thereof in treating diseases, in particular cancer.

### FIELD OF THE INVENTION

The invention is in the field of immunotherapy and relates to humanized anti-Nectin-4 antibodies and to antibody-conjugates and therapeutic and diagnostic compositions comprising them, for treating diseases, in particular cancer.

### BACKGROUND OF THE INVENTION

Cancer immunotherapy is utilized for generating and augmenting an anti-tumor immune response, e.g., by treatment with antibodies specific to antigens on tumor cells, or by specific activation of anti-tumor T cells. The ability of recruiting immune cells (e.g., T cells) against tumor cells in a patient provides a therapeutic modality of fighting cancer types and metastasis that are otherwise considered incurable.

Nectin Cell Adhesion Molecule 4 (Nectin-4), also termed poliovirus receptor-related 4 (PVRL4), is a type I transmembrane protein and member of the Nectin family of related immunoglobulin-like adhesion molecules. Nectin-4 is a tumor associated marker for many tumors including bladder cancer, breast cancer, lung cancer and other malignancies.

Chailta-Eid at al. 2016 (Cancer Res. 2016; 76:3003-13) disclose anti Nectin-4 (Enfortumab) antibody-drug conjugate as a highly potent therapeutic agent in multiple preclinical cancer models. The antibody, conjugated with the microtubule inhibitor vedotin, binds human, as well as rat and monkey Nectin-4 and inhibits growth of several cell lines and xenografts that express Nectin-4.

International patent application publication No. WO 2019/215728 discloses monoclonal antibodies that recognize human Nectin-4 with high affinity and specificity and inhibit its binding to T cell immunoreceptor with Ig and ITIM domains (TIGIT).

Antibody-drug conjugates (ADCs) are a promising tool for both direct tumor cell killing and for the consequent activation of bystander immune cells. These therapeutic entities are composed of mAbs linked to cytotoxic drugs (payloads), and are designed, in principle, to widen the therapeutic window of those drugs by limiting their delivery specifically to cells that express the target antigen, and thus to reduce their systemic exposure and toxicity.

Auristatin is a microtubule-destroying drug. It was derived from marine shell-less mollusk Dolabella auricularia called dolastatins. Various derivatives of auristatin have been synthesized, such as monomethyl auristatin E (MMAE) and monomethyl auristatin F (MMAF). MMAE and MMAF were developed by Seattle Genetics and used as payloads for ADCs. MMAF and MMAE have their advantages and disadvantages. MMAE is more membrane-permeable and has a lower IC50 than MMAF. However, MMAF is more hydrophilic and has a lower aggregation tendency to show lower systemic toxicity than MMAE (park et al. Molecules 2019, 24, 2754).

International patent application publication No. WO 2018/158398 discloses antibodies having specificity to Nectin-4, such as 14A5.2, and their uses in treating cancer.

International patent application publication No. WO 2017/042210 discloses anti Nectin-4 antibody conjugated to MMAE which is more efficient than enfortumab-vedotin (PADCEV). The antibodies used in this application are of murine origin, making them less suitable for clinical use.

There is an unmet need to provide humanized antibodies recognizing human Nectin-4 and ADCs comprising them, that are improved, safe and potent and that may be useful in treating cancers expressing Nectin-4.

### SUMMARY OF THE INVENTION

The present invention provides humanized antibodies that specifically bind Nectin-4, or antigen binding portion thereof. The humanized antibodies of the present invention, selected from a larger collection of antibody clones, have improved properties compared to other anti-Nectin-4 antibodies. The present invention further provides, according to some embodiments, conjugates comprising the antibodies and therapeutic or diagnostic agents. In some embodiments, the conjugates comprise a cytotoxic moiety and are useful in treating cancer having tumor cells presenting the Nectin-4 receptor on their surface.

Notably, the present invention demonstrates that the NTX1105 ADC is more efficient than the most advanced ADC bearing an anti-Nectin-4 antibody published so far (PADCEV or enfortumab-based ADCs). It is now shown that NTX1105-based ADCs have significant anti-tumor activity in treatment regiments in which PADCEV, or other enfortumab-based ADCs have no activity.

A large collection of humanized antibodies was produced by combining specific sets of complementarity determining regions (CDR) sequences and human framework sequences and introducing specific mutations in these sequences to produce antibodies with modified variable regions and improved properties. The antibodies disclosed herein were designed based on factors including homology, T-cell epitopes, key residues, and predicted structures. Advantageously, the newly designed humanized variable regions described herein preserve the residues critical for the maintenance of the antibody's conformation and binding affinity, while having significantly lower incidence of potential T cell epitopes, thus minimizing the risk of adverse immune response towards the antibodies.

The humanized antibodies disclosed herein had superior productivity characteristic, and improved developability properties (e.g higher resistance to aggregation as measured by improved Tagg). The humanized antibodies disclosed herein were found to be highly suitable for use as targeted therapy with therapeutic toxins. It is now disclosed that the anti-Nectin-4 monoclonal humanized antibodies described herein, conjugated to a cytotoxic moiety, exhibit robust killing of various tumor cell lines. The direct targeting of toxins using the antibodies described herein has the potential to increase the anti-tumor activity of these toxins and to improve the survival of cancer patients. Some of the ADCs of the present invention comprise sequence modification of their Fc region that significantly reduces their binding by FcγR-bearing normal cells, thereby increasing their safety.

According to one aspect, the present invention provides a humanized antibody that specifically binds human Nectin-4, or a fragment thereof comprising at least the antigen binding site, wherein the antibody or a fragment thereof comprises a heavy chain and a light chain, wherein the heavy chain comprises a variable region having an amino acid sequence at least about 90% identical to a sequence selected from the group consisting of SEQ ID NO: 2, SEQ ID NO: 3, SEQ ID NO: 4, SEQ ID NO: 5 and SEQ ID NO: 6; and wherein the light chain comprises a variable region having an amino acid sequence at least about 90% identical to a sequence selected from the group consisting of SEQ ID NO: 8, SEQ ID NO: 9, SEQ ID NO: 10, SEQ ID NO: 11 and SEQ ID NO: 12.

According to some embodiments, the humanized antibody or a fragment thereof comprises a heavy chain comprising a variable region having an amino acid sequence at least about 95% identical to SEQ ID NO: 2. According to some embodiments, the humanized antibody or a fragment thereof comprises a heavy chain comprising a variable region having an amino acid sequence at least about 95% identical to SEQ ID NO: 3. According to some embodiments, the humanized antibody or a fragment thereof comprises a heavy chain comprising a variable region having an amino acid sequence at least about 95% identical to SEQ ID NO: 4. According to some embodiments, the humanized antibody or a fragment thereof comprises a heavy chain comprising a variable region having an amino acid sequence at least about 95% identical to SEQ ID NO: 5. According to some embodiments, the humanized antibody or a fragment thereof comprises a heavy chain comprising a variable region having an amino acid sequence at least about 95% identical to SEQ ID NO: 6.

According to some embodiments, the humanized antibody or a fragment thereof comprises a light chain comprising a variable region having an amino acid sequence at least about 95% identical to SEQ ID NO: 8. According to some embodiments, the humanized antibody or a fragment thereof comprises a light chain comprising a variable region having an amino acid sequence at least about 95% identical to SEQ ID NO: 9. According to some embodiments, the humanized antibody or a fragment thereof comprises a light chain comprising a variable region having an amino acid sequence at least about 95% identical to SEQ ID NO: 10. According to some embodiments, the humanized antibody or a fragment thereof comprises a light chain comprising a variable region having an amino acid sequence at least about 95% identical to SEQ ID NO: 11. According to some embodiments, the humanized antibody or a fragment thereof comprises a light chain comprising a variable region having an amino acid sequence at least about 95% identical to SEQ ID NO: 12.

According to some embodiments, the humanized antibody or a fragment thereof comprises a heavy chain and a light chain, wherein the heavy chain comprises a variable region having an amino acid sequence at least about 95% identical to SEQ ID NO: 13, and the light chain comprises a variable region having an amino acid sequence at least about 95% identical to SEQ ID NO: 14.

According to some embodiments, the humanized antibody or a fragment thereof comprises a heavy chain comprising a variable region having the amino acid set forth in SEQ ID NO: 13. According to some embodiments, the humanized antibody or a fragment thereof comprises a light chain comprising a variable region having the amino acid set forth in SEQ ID NO: 14. According to some embodiments, the humanized antibody or a fragment thereof comprises a heavy chain and a light chain, wherein the heavy-chain variable region comprises the sequence set forth in SEQ ID NO: 13, and the light-chain variable region comprises the sequence set forth in SEQ ID NO: 14.

There are several methods known in the art for determining the CDR sequences of a given antibody molecule, but there is no standard unequivocal method. Determination of CDR sequences from antibody heavy and light chain variable regions can be made according to any method known in the art, including but not limited to the methods known as KABAT, Chothia and IMGT. A selected set of CDRs may include sequences identified by more than one method, namely, some CDR sequences may be determined using KABAT and some using IMGT, for example. According to some embodiments, the CDR sequences of the mAb variable regions are determined using the IMGT method.

According to some embodiments, the humanized antibody or a fragment thereof comprises a set of six CDR sequences, wherein the heavy-chain CDR1 comprising the sequence SYY (SEQ ID NO: 26), heavy-chain CDR2 comprising the sequence IYPGNVNT (SEQ ID NO: 22), heavy-chain CDR3 comprising the sequence SNPYVMDY (SEQ ID NO: 17), light-chain CDR1 comprising the sequence QSVNND (SEQ ID NO: 24), light-chain CDR2 comprising the amino acid sequence YAS (SEQ ID NO: 25), and light-chain CDR3 comprising the sequence QQAYRSPYT (SEQ ID NO: 20).

According to some embodiments, the humanized antibody or a fragment thereof comprises a set of six CDR sequences, wherein heavy-chain CDR1 comprising the sequence SYYIH (SEQ ID NO: 15), heavy-chain CDR2 comprising the sequence WIYPGNVNTKYNERF(K/Q)G (SEQ ID NO: 16), heavy-chain CDR3 comprising the sequence SNPYVMDY (SEQ ID NO: 17), light-chain CDR1 comprising the sequence (K/R)ASQSVNNDVA (SEQ ID NO: 18), light-chain CDR2 comprising the sequence YASNRFT (SEQ ID NO: 19), and light-chain CDR3 comprising the sequence QQAYRSPYT (SEQ ID NO: 20).

According to some embodiments, the humanized antibody or a fragment thereof comprises a heavy-chain CDR2 comprising the sequence WIYPGNVNTKYNERFKG (SEQ ID NO: 27). According to some embodiments, the humanized antibody or a fragment thereof comprises a heavy-chain CDR2 comprising the sequence WIYPGNVNTKYNERFQG (SEQ ID NO: 28).

According to some embodiments, the humanized antibody or a fragment thereof comprises a light-chain CDR1 comprising the sequence KASQSVNNDVA (SEQ ID NO: 29). According to some embodiments, the humanized antibody or a fragment thereof comprises a light-chain CDR1 comprising the sequence RASQSVNNDVA (SEQ ID NO: 30).

According to some embodiments, the humanized antibody or a fragment thereof comprises a set of six CDR sequences, wherein heavy-chain CDR1 comprising the sequence GYTFTSYY (SEQ ID NO: 21), heavy-chain CDR2 comprising the sequence IYPGNVNT (SEQ ID NO: 22), heavy-chain CDR3 comprising the sequence ARSNPYVMDY (SEQ ID NO: 23), Light-chain CDR1 comprising the sequence QSVNND (SEQ ID NO: 24), Light-chain CDR2 comprising the amino acid sequence YAS (SEQ ID NO: 25), and Light-chain CDR3 comprising the sequence QQAYRSPYT (SEQ ID NO: 20).

According to some embodiments, the humanized antibody or antigen binding fragment thereof comprising: a heavy chain variable region and a light chain variable region, the heavy chain variable region comprising a set of four heavy chain (HC) framework (FR) sequences: (A) FR-H1 selected from the group consisting of SEQ ID NOs: 31, 32, and 33; (B) FR-H2 selected from the group consisting of SEQ ID NOs: 34, 35, and 36; (C) FR-H3 selected from the group consisting of SEQ ID NOs: 37, 38, 39, and 40; (D) FR-H4 is SEQ ID NO: 41; and the light chain variable region comprising a set of four light chain (LC) framework (FR) sequences: (A) FR-L1 selected from the group consisting of SEQ ID NOs: 42, 43, and 44; (B) FR-L2 selected from the group consisting of SEQ ID NOs: 45 and 46; (C) FR-L3 selected from the group consisting of SEQ ID NOs: 47, 48 and 49; and (D) FR-L4 is SEQ ID NO: 50. Each possibility or combination of framework represents a separate embodiment.

According to some embodiments, the heavy chain variable region of the humanized monoclonal antibody comprises an amino acid sequence at least about 97% identical to a sequence selected from the group consisting of SEQ ID NOs: 2, 3, 4, 5, and 6; and the light chain variable region of the humanized monoclonal antibody comprises an amino acid sequence at least about 97% identical to a sequence selected from the group consisting of SEQ ID NOs: 8, 9, 10, 11, and 12. Each possibility or combination of heavy and light chains represents a separate embodiment of the invention.

According to some embodiments, the heavy chain variable region of the humanized monoclonal antibody comprises a sequence selected from the group consisting of SEQ ID NOs: 2, 3, 4, 5, and 6; and the light chain variable region of the humanized monoclonal antibody comprises a sequence selected from the group consisting of SEQ ID NOs: 8, 9, 10, 11, and 12. Each combination of heavy and light chain variable region represents a separate embodiment of the invention. According to certain exemplary embodiments, the heavy chain variable region of the humanized monoclonal antibody comprises the SEQ ID NO: 5; and the light chain variable region of the humanized monoclonal antibody comprises the SEQ ID NO: 12 (denoted herein NTX1105 (H4/k5)). According to additional exemplary embodiments, the heavy chain variable region of the humanized monoclonal antibody comprises the SEQ ID NO: 6; and the light chain variable region of the humanized monoclonal antibody comprises the SEQ ID NO: 12.

According to some embodiments, the humanized antibody or fragment thereof is a monoclonal antibody. Fab, F (ab) 2, a single-domain antibody, or a single chain variable fragment (scFv).

According to some embodiments, the humanized antibody or fragment thereof is an IgG monoclonal antibody. According to some embodiments, the humanized antibody has a heavy chain constant region selected from IgG1, IgG4, and IgG2. In certain embodiments, the humanized antibody or fragment thereof is an IgG4 subclass. In certain embodiments, the humanized antibody or antigen binding fragment thereof is an IgG1 subclass. According to some embodiments the antibody has a kappa light chain constant region.

According to some embodiment, the humanized antibody has a mutated Fc domain that prevents FcγR-mediated internalization.

According to some embodiments, the humanized antibody comprises a Fc null domain. According to certain embodiments, the Fc domain is null for binding to Fcγ receptors found on immune cells. According to certain exemplary embodiments, the Fc domain is null for binding to CD64, CD32a, CD32b, CD16a, and/or CD16b.

According to some embodiments, the humanized antibody comprises a Fc null domain having the LALAPG mutant.

According to some embodiments, the humanized antibody comprising a heavy chain sequence set forth in SEQ ID NO: 51, and a light chain sequence set forth in SEQ ID NO: 52 (NTX1105 (H4/k5) having LALAPG mutant (FcgR^{null})).

According to some embodiments, a conjugate comprising the humanized antibody or fragment thereof described above is provided.

Antibodies or fragments thereof according to the present invention may be attached to a cytotoxic moiety, a radioactive moiety, or a labeling tag.

According to some embodiments, the humanized antibody or fragment thereof is conjugated to a toxin (payload).

According to some embodiments, the toxin is selected from the group consisting of microtubule inhibitor, DNA synthesis inhibitor, topoisomerase inhibitor, and RNA polymerase inhibitor.

According to certain embodiments, the toxin is a microtubule-destroying drug. According to certain exemplary embodiments, the toxin is auristatin or a derivative thereof. According to certain embodiments, the auristatin derivative is monomethyl auristatin E (MMAE) or monomethyl auristatin F (MMAF).

According to some embodiments, the toxin is saporin.

According to some embodiments, the toxin is a maytansine derivative. According to certain embodiments, the maytansine derivative is DM4 or DM1.

According to some embodiments, the toxin is quinoline alkaloid. According to certain embodiments, the quinoline alkaloid is SN-38.

According to some embodiments, the toxin is directly linked to the antibody. According to other embodiments, the antibody and the toxin are linked through a linker. According to some embodiments, the toxin is covalently linked to the humanized antibody directly or through a linker.

According to some embodiments, the linker is cleavable. According to additional embodiments, the linker is not cleavable. According to some embodiments, the linker is an enzymatic cleavable linker. According to certain embodiments, the linker is a pH-sensitive linker. According to some embodiments, the linker is a reducible linker (sulfo-SPDB).

According to some embodiments, the linker is selected from the group consisting of Maleimidocaproyl (MC), Maleimidocaproyl-Valine-Citrulline-p-amino-benzyloxycarbonyl (MC-VC-PAB), Maleimidomethyl cyclohexane-1-carboxylate (SMCC), N-succinimidyl-4-(2-pyridyldithio) butanoate (SPDB) and Lys-PAB-CO (Lysine-ρ-aminobenzyl-C═O).

According to some embodiments, the Drug-to-Antibody Ratio (DAR) is 4-8. According to certain embodiments, the DAR is 4 (DAR-4). According to certain embodiments, the DAR is 8 (DAR-8).

According to some embodiments, polynucleotide sequences encoding the amino acid sequences of the heavy chain variable region and the light chain variable region as described above are provided.

In a further aspect, the present invention provides a nucleic acid construct comprising a nucleic acid molecule encoding at least one humanized antibody chain or fragment thereof as described herein. According to some embodiments the nucleic acid construct is a plasmid.

According to some embodiments, there is provided a plasmid for expressing the humanized antibodies or fragment thereof as described herein, the plasmid comprising nucleic acid molecule encoding the antibody.

The present invention provides, according to another aspect, a pharmaceutical composition comprising the humanized antibody or antigen binding fragment described herein or a conjugate comprising the antibody and a pharmaceutically acceptable excipient, carrier, or diluent.

According to some embodiments, the pharmaceutical composition is for use in treating cancer.

Any administration mode may be used to deliver the compositions of the present invention to a subject in need thereof, including parenteral and enteral administration modes.

According to some embodiments, the pharmaceutical composition is formulated for injection or infusion. According to some embodiments, the pharmaceutical composition is formulated for intravenous administration. In certain embodiments, the pharmaceutical composition is formulated for intratumoral administration.

According to yet another aspect, the present invention provides a method of treating cancer comprising administering to a subject in need thereof, a therapeutically effective amount of at least one humanized antibody, a fragment thereof or their conjugate as described herein.

According to some embodiments, the cancer comprises a solid tumor.

According to certain embodiments, the cancer is selected from the group consisting of prostate cancer, colorectal cancer, bladder cancer, liver cancer, ovarian cancer, endometrial cancer, stomach cancer, thyroid cancer, carcinoid tumor, head and neck cancer, breast cancer, pancreatic cancer, testis cancer, urothelial cancer, cervical cancer, melanoma, lymphoma and lung cancer. Each possibility represents a separate embodiment of the invention.

According to certain embodiments, the cancer is selected from the group consisting of Pancreatic Ductal Adenocarcinoma, Kidney Renal Clear Cell Carcinoma and Skin Cutaneous Melanoma.

According to other embodiments, the cancer is a hematological cancer. According to some embodiments, the hematological cancer is selected from leukemia including acute myeloid leukemia (AML), chronic myeloid leukemia (CML), acute lymphocytic leukemia (ALL), and chronic lymphocytic leukemia (CLL); lymphoma, including Hodgkin disease, and non-Hodgkin lymphoma; and multiple myeloma.

According to some embodiments, the subject is human.

According to some embodiments, the method of treating cancer comprises administering or performing at least one additional anti-cancer therapy. According to certain embodiments, the additional anti-cancer therapy is surgery, chemotherapy, radiotherapy, or immunotherapy.

According to some embodiments, the method of treating cancer comprises administration of the humanized antibody or conjugate described herein and an additional anti-cancer agent. According to some embodiments, the additional anti-cancer agent is selected from the group consisting of: immune-modulator, activated lymphocyte cell, kinase inhibitor and chemotherapeutic agent.

According to some embodiments, the anti-cancer agent is selected from the group consisting of: erbitux, cytarabine, fludarabine, fluorouracil, mercaptopurine, methotrexate, thioguanine, gemcitabine, vincristine, vinblastine, vinorelbine, carmustine, lomustine, chlorambucil, cyclophosphamide, cisplatin, carboplatin, ifosfamide, mechlorethamine, melphalan, thiotepa, dacarbazine, bleomycin, dactinomycin, daunorubicin, doxorubicin, idarubicin, mitomycin, mitoxantrone, plicamycin, etoposide, teniposide and any combination thereof. Each possibility represents a separate embodiment of the invention.

According to some embodiments, the method of treating cancer involves preventing or reducing formation, growth or spread of metastases in a subject.

According to another aspect, the present invention provides an antibody-drug conjugate (ADC) comprising a humanized anti-Nectin-4 antibody or antigen binding portion thereof, conjugated to a toxin (payload), the antibody or antigen binding portion thereof comprises a set of six CDR sequences, wherein heavy-chain CDR1 comprising the sequence SYY (SEQ ID NO: 26), heavy-chain CDR2 comprising the sequence IYPGNVNT (SEQ ID NO: 22), heavy-chain CDR3 comprising the sequence SNPYVMDY (SEQ ID NO: 17), light-chain CDR1 comprising the sequence QSVNND (SEQ ID NO: 24), light-chain CDR2 comprising the amino acid sequence YAS (SEQ ID NO: 25), and light-chain CDR3 comprising the sequence QQAYRSPYT (SEQ ID NO: 20).

According to some embodiments, the antibody-drug conjugate (ADC) comprises a humanized anti-Nectin-4 antibody or antigen binding portion thereof, comprising a set of six CDR sequences, wherein heavy-chain CDR1 comprising the sequence SYYIH (SEQ ID NO: 15), heavy-chain CDR2 comprising the sequence WIYPGNVNTKYNERF(K/Q)G (SEQ ID NO: 16), heavy-chain CDR3 comprising the sequence SNPYVMDY (SEQ ID NO: 17), light-chain CDR1 comprising the sequence (K/R)ASQSVNNDVA (SEQ ID NO: 18), light-chain CDR2 comprising the sequence YASNRFT (SEQ ID NO: 19), and light-chain CDR3 comprising the sequence QQAYRSPYT (SEQ ID NO: 20).

According to some embodiments, the humanized antibody or a fragment thereof comprises a heavy-chain CDR2 comprising the sequence WIYPGNVNTKYNERFKG (SEQ ID NO: 27). According to some embodiments, the humanized antibody or a fragment thereof comprises a heavy-chain CDR2 comprising the sequence WIYPGNVNTKYNERFQG (SEQ ID NO: 28).

According to some embodiments, the humanized antibody or a fragment thereof comprises a light-chain CDR1 comprising the sequence KASQSVNNDVA (SEQ ID NO: 29). According to some embodiments, the humanized antibody or a fragment thereof comprises a light-chain CDR1 comprising the sequence RASQSVNNDVA (SEQ ID NO: 30).

According to some embodiments, the antibody or a fragment thereof comprises a heavy chain and a light chain, wherein the heavy chain comprises a variable region having an amino acid sequence at least about 90% identical to a sequence selected from the group consisting of SEQ ID NO: 2, SEQ ID NO: 3, SEQ ID NO: 4, SEQ ID NO: 5 and SEQ ID NO: 6; and wherein the light chain comprises a variable region having an amino acid sequence at least about 90% identical to a sequence selected from the group consisting of SEQ ID NO: 8, SEQ ID NO: 9, SEQ ID NO: 10, SEQ ID NO: 11 and SEQ ID NO: 12.

According to some embodiments, the heavy chain variable region of the humanized antibody comprises an amino acid sequence set forth in SEQ ID NO. 13; and the light chain variable region of the humanized antibody comprises an amino acid sequence set forth in SEQ ID NO: 14.

According to some embodiments, the heavy chain variable region of the humanized antibody comprises an amino acid sequence at least about 97% identical to a sequence selected from the group consisting of SEQ ID NOs: 2, 3, 4, 5, and 6; and the light chain variable region of the humanized monoclonal antibody comprises an amino acid sequence at least about 97% identical to a sequence selected from the group consisting of SEQ ID NOs: 8, 9, 10, 11, and 12.

According to some embodiments, the heavy chain variable region of the humanized monoclonal antibody comprises a sequence set forth in SEQ ID NO: 5, and the light chain variable region of the humanized monoclonal antibody comprises a sequence set forth in SEQ ID NO: 12. According to other embodiments, the heavy chain variable region of the humanized monoclonal antibody comprises a sequence set forth in SEQ ID NO: 6, and the light chain variable region of the humanized monoclonal antibody comprises a sequence set forth in SEQ ID NO: 12.

According to some embodiments, the antibody-drug conjugate comprises a toxin as described hereinabove.

Any chemical or biological entity that capable of killing or inhibiting the growth of tumor cells in vivo may be used with the ADCs of the present invention as a toxin. According to some embodiments, the toxin is selected from the group consisting of microtubule inhibitor, DNA synthesis inhibitor, topoisomerase inhibitor, and RNA polymerase inhibitor.

According to certain embodiments, the toxin is a microtubule-destroying drug. According to certain exemplary embodiments, the toxin is auristatin or a derivative thereof. According to certain embodiments, the auristatin derivative is monomethyl auristatin E (MMAE) or monomethyl auristatin F (MMAF).

According to some embodiments, the toxin is saporin.

According to some embodiments, the toxin is a maytansine derivative. According to certain embodiments, the maytansine derivative is DM4 or DM1.

According to some embodiments, the toxin is a quinoline alkaloid. According to certain embodiments, the quinoline alkaloid is SN-38.

According to some embodiments, the toxin is selected from the group consisting of DM4, MMAE and SN-38. According to certain embodiments, the toxin is DM4 or MMAE.

According to some embodiments, the toxin is a topoisomerase I inhibitor. According to some embodiments, the toxin is a derivative of camptothecin. According to certain embodiments, the toxin is Exatecan.

According to some embodiments, the toxin is directly linked to the antibody. According to other embodiments, the antibody and the toxin are linked through a linker. According to some embodiments, the toxin is covalently linked to the humanized antibody directly or through a linker.

According to some embodiments, the linker is cleavable. According to other embodiments, the linker is not cleavable. According to some embodiments, the cleavable linker is selected from the group consisting of an enzymatic cleavable linker, a pH-sensitive linker and a reducible linker. According to some embodiments, the linker is an enzymatic cleavable linker. According to certain embodiments, the linker is a pH-sensitive linker. According to some embodiments, the linker is a reducible linker.

According to some embodiments, the linker is selected from the group consisting of Maleimidocaproyl (MC), Maleimidocaproyl-Valine-Citrulline-p-amino-benzyloxycarbonyl (MC-VC-PAB), Maleimidomethyl cyclohexane-1-carboxylate (SMCC), N-succinimidyl-4-(2-pyridyldithio) butanoate (sulfo-SPDB), a valine-alanine linker and Lys-PAB-CO (Lysine-ρ-aminobenzyl-C═O).

According to some embodiments, the conjugate comprises the toxin MMAE and the linker MC-VC-PAB (denoted herein NTX1105-MMAE). According to some embodiments, the conjugate comprises the toxin MMAF and the linker MC (denoted herein NTX1105-MMAF). According to some embodiments, the conjugate comprises the toxin DM1 and the linker SMCC (denoted herein NTX1105-DM1). According to some embodiments, the conjugate comprises the toxin DM4 and the linker SPDB (denoted herein NTX1105-DM4). According to some embodiments, the conjugate comprises the toxin SN38 and the linker Lys-PAB-CO (denoted herein NTX1105-SN38).

According to some embodiments, the ADC comprises an anti-Nectin-4 antibody that competes with an antibody described herein to specifically bind to the Nectin-4 molecule. According to certain embodiments, the ADC comprises an anti-Nectin-4 antibody that competes with an antibody comprising heavy-chain and light chain variable regions wherein heavy-chain CDR1 comprising the sequence SYYIH (SEQ ID NO: 15), heavy-chain CDR2 comprising the sequence WIYPGNVNTKYNERF(K/Q)G (SEQ ID NO: 16), heavy-chain CDR3 comprising the sequence SNPYVMDY (SEQ ID NO: 17), light-chain CDR1 comprising the sequence (K/R)ASQSVNNDVA (SEQ ID NO: 18), light-chain CDR2 comprising the sequence YASNRFT (SEQ ID NO: 19), and light-chain CDR3 comprising the sequence QQAYRSPYT (SEQ ID NO: 20), to specifically bind to the Nectin-4 molecule.

The present invention provides, according to another aspect, a pharmaceutical composition comprising the antibody-drug conjugate described herein and a pharmaceutically acceptable excipient, carrier, or diluent.

Any administration mode may be used to deliver the compositions of the present invention to a subject in need thereof, including parenteral and enteral administration modes.

According to some embodiments, the pharmaceutical composition is formulated for injection or infusion. According to some embodiments, the pharmaceutical composition is formulated for intravenous (IV) administration. In certain embodiments, the pharmaceutical composition is formulated for intratumoral (IT) administration.

According to some embodiments, the conjugate or the pharmaceutical composition is for use in treating a cancer in an individual.

The cancer is as described hereinabove. In certain embodiments, the cancer comprises a solid tumor. In certain embodiments, the cancer is selected from the group consisting of liver cancer, lung cancer, colon cancer, glioblastoma, adrenal cancer, uterine cancer, testis cancer, head and neck cancer, pancreatic cancer, and breast cancer. Each possibility represents a separate embodiment of the invention.

According to some embodiments of the invention, the use further comprises the use in a combination with an additional ADC.

The present invention provides, according to another aspect, a method of treating a cancer in an individual in need of such treatment, the method comprising administering to the individual a therapeutically effective amount of the conjugate or the pharmaceutical composition described herein. In certain embodiments, the cancer is a solid tumor. According to additional embodiments, the cancer is a non-solid tumor. In certain embodiments, the cancer is selected from the group consisting of glioblastoma, pancreatic cancer, breast cancer, bladder cancer, kidney cancer, head and neck cancer, ovarian cancer, colon cancer, cervical cancer, prostate cancer, and lung cancer. In certain embodiments, the method of treating cancer involves preventing or reducing formation, growth or spread of metastases in a subject.

According to other embodiments, the cancer is a hematological cancer. According to some embodiments, the hematological cancer is selected from leukemia including acute myeloid leukemia (AML), chronic myeloid leukemia (CML), acute lymphocytic leukemia (ALL), and chronic lymphocytic leukemia (CLL); lymphoma, including Hodgkin disease, and non-Hodgkin lymphoma; and multiple myeloma.

According to some embodiments, the individual is human.

According to some embodiments, the method of treating cancer comprises administering or performing at least one additional anti-cancer therapy. According to certain embodiments, the additional anticancer therapy is surgery, chemotherapy, radiotherapy, or immunotherapy.

According to some embodiments, the method of treating cancer comprises administration of the conjugate described herein and an additional anti-cancer agent. According to some embodiments, the additional anti-cancer agent is selected from the group consisting of: immune-modulator, activated lymphocyte cell, kinase inhibitor and chemotherapeutic agent.

Also described herein is a method of making composition for treating a cancer in an individual afflicted with cancer comprising admixing the ADCs described herein and a pharmaceutically acceptable excipient, carrier, or diluent.

According to yet another aspect, the present invention provides a method of delivering at least one humanized antibody, a fragment thereof or conjugate as described herein to a cell comprising contacting the cell with the humanized antibody, a fragment thereof or conjugate.

According to some embodiments, the method comprises administering the humanized antibody, a fragment thereof or conjugate to cells of a subject. According to certain embodiments, the subject is a human subject.

According to another aspect, the present invention provides a method of delivering at least one humanized antibody, a fragment thereof or their conjugate as described herein to a cell of a subject, comprising administering the at least one humanized antibody, a fragment thereof or conjugate to the subject. According to some embodiments, the cell is a tumor cell.

The present invention further provides, according to an aspect, a method of diagnosing or prognosing cancer in a subject, the method comprises determining the expression level of Nectin-4 in a biological sample of said subject using at least one antibody conjugate as described herein.

According to some embodiments, the method comprising detecting the antibody or antibody fragment that is bound, and determining the levels of expression of Nectin-4 in the sample. According to certain embodiments, the method comprises comparing the levels of expression to control. According to some embodiments, the control is a predefined value. According to certain embodiments, the control is a corresponding non-cancerous tissue. According to some embodiments, the comparison indicates or suggests if the subject has cancer.

According to some embodiments, the method is used for diagnosis of cancer subtype. According to some embodiments, the method is used for determine patient eligibility to receive an anti-cancer therapy. According to certain embodiments, the anti-cancer therapy is an antibody-drug conjugate. According to some embodiments, the method is used for determine patient eligibility to receive a therapy with an ADC as described herein.

The present invention further provides, according to another aspect, a method of diagnosing, determining or quantifying the expression of Nectin-4, the method comprising contacting a biological sample with an antibody conjugate as described herein, and measuring the level of complex formation.

According to some embodiments, the method for detecting or quantifying the expression of Nectin-4 comprises the steps of:
i. incubating a sample with the antibody conjugate described herein;
ii. detecting the bound Nectin-4 using said conjugate.

According to some embodiments, the method further comprises the steps of:
iii. comparing the amount of step (ii) to a standard curve obtained from a reference sample containing a known amount of Nectin-4; and
iv. calculating the amount of the Nectin-4 in the sample from the standard curve.

According to some particular embodiments, the sample is a body fluid or solid tissue. In some embodiments, the method is performed in-vitro or ex-vivo.

A kit for measuring the expression of Nectin-4 in a biological sample is also provided comprising at least one conjugate as described herein and means for measuring Nectin-4 expression. In some embodiment, the kit further comprising instruction material directing the use of the kit.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1C show the correlation of Nectin-4 expression mRNA levels (high or low as indicated) with survival probability of: Pancreatic Ductal Adenocarcinoma (FIG. 1A), Kidney Renal Clear Cell Carcinoma (FIG. 1B) and Skin Cutaneous Melanoma. (FIG. 1C) patients. Data sets were obtained from the TCGA site and analyzed using oncolnc.org site (https://doi.org/10.7717/peerj-cs.67). N depicts number of patients included at the analysis.

FIG. 2 shows a graph showing percent of tumors positive for Nectin-4 expression. Data obtained from proteinatlas.com using the HPA010775 mAb (anti Nectin-4 Sigma-Aldrich®). In 13/20 indications moderate-high membranous expression of Nectin-4 is seen.

FIG. 3 depicts a dose dependent killing of MDA-MB-468 cell line (breast adenocarcinoma cells) in-vitro using the various linker-payload combinations of NTX1105 (VH0/Vκ0).

FIG. 4 depicts in-vivo efficacy of the NTX1105 (VH0/Vκ0) ADCs against the Triple-negative breast cancer (TNBC) tumor cells line MDA-MB-468 that were implanted subcutaneous (s.c.) to NOD-SCID female mice. The effect of treatment with all NTX1105 combinations of linker payloads (LP) is shown.

FIG. 5 demonstrates improved characteristics for some of the humanized variants. The productivity of the parental/chimeric (VH0/Vκ0) antibody and six purified lead humanized variants was evaluated.

FIGS. 6A-6B show robust in-vivo activity of the humanized variants. In-vivo efficacy of the lead humanized NTX1105 (VH4/Vκ5 and VH5/Vκ5) ADCs, both conjugated to MC-VC-PAB-MMAE at DAR˜4, against the TNBC tumor cells line, MDA-MB-468, was evaluated. The results are shown as tumor volume (FIG. 6A) and as a fold change of the tumor volume along time (FIG. 6B).

FIGS. 7A-7B show differential in-vitro activity of NTX1105 (H4K5) ADC compared to Enfortumab-vedotin (PADCEV). Both ADCs were tested for on-cell binding (FIG. 7A) and in-vitro killing activity (FIG. 7B).

FIGS. 8A-8B show that the anti-tumor activity in-vivo of NTX1105 (H4K5) conjugated to MC-VC-PAB-MMAE, at DAR˜4 is superior to Enfortumab-vedotin (PADCEV). To compare the ADC to PADCEV, the activity of both ADCs was evaluated in-vivo, using H322 (NSCLC model). The ADCs were given either at 6-doses of 1 mg/kg (FIG. 8A) or 3 doses of 3 mg/kg (FIG. 8B).

FIG. 9 depicts in-vivo efficacy of humanized NTX1105 (H4κ5-FcgR^{null}) ADCs with either DAR-4 MMAE or DAR-8 Exatecan payload against H322M (lung cancer model) s.c. tumor model in Nude female mice.

FIG. 10 depicts in-vivo superiority of humanized NTX1105 (H4κ5-FcgR^{null}) anti-Nectin-4 ADCs with DAR-4 Exatecan compared to Enfortumab-DAR-4 Exatecan against large (>450 mm³) H322M. tumor model in Nude female mice.

### DETAILED DESCRIPTION

The present invention provides humanized anti-Nectin-4 antibodies and antibody-drug conjugates, or ADCs, comprising them, which are useful in treating cancer. Advantageously, the ADCs described herein comprise antibodies that are almost fully humanized, thus avoiding the risk of adverse immune response towards the antibodies and are therefore likely to be safe for use in humans. The ADCs described herein were found to be highly potent and better than other known anti-Nectin-4 ADCs.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a.” “an.” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments. As used herein the term “about” refers to an amount that is near the stated amount by 10% or less.

The term “Nectin-4” or “Nectin Cell Adhesion Molecule 4”, as used herein refers to a single-pass type I membrane protein of 510 amino acids and a molecular mass of 55454 Da, also known as PVRL4; LNIR; PRR4; and EDSS1. The Nectin-4 protein contains two immunoglobulin-like (Ig-like) C2-type domains and one Ig-like V-type domain. It is involved in cell adhesion through trans-homophilic and -heterophilic interactions. The soluble form is produced by proteolytic cleavage at the cell surface by the metalloproteinase ADAM17/TACE and the secreted form is found in both breast tumor cell lines and breast tumor patients. An exemplary Nectin-4 according to the invention is set forth in SwissPort, UniPort and GenBank symbols or accession numbers: Q96NY8-NECT4_HUMAN; Q96NY8; B4DQW3; Q96K15; Q96NY8-1; Q96NY8-2; ENSP00000356991; NP_112178.2; XP_005245565.1; XP_011508323.1; XP_011508324.1; or XP_011508325.1.

According to one aspect, the present invention provides a humanized antibody that specifically binds human Nectin-4, or a fragment thereof comprising at least the antigen binding site, wherein the antibody or a fragment thereof comprises a heavy chain and a light chain, wherein the heavy chain comprises a variable region having an amino acid sequence at least about 90% identical to a sequence selected from the group consisting of SEQ ID NO: 2, SEQ ID NO: 3, SEQ ID NO: 4, SEQ ID NO: 5 and SEQ ID NO: 6; and wherein the light chain comprises a variable region having an amino acid sequence at least about 90% identical to a sequence selected from the group consisting of SEQ ID NO: 8, SEQ ID NO: 9, SEQ ID NO: 10, SEQ ID NO: 11 and SEQ ID NO: 12.

According to another aspect, the present invention provides an antibody-drug conjugate (ADC) comprising a humanized anti-Nectin-4 antibody or antigen binding portion thereof, conjugated to a toxin (payload), the antibody or antigen binding portion thereof comprises a set of six CDR sequences, wherein heavy-chain CDR1 comprising the sequence SYY (SEQ ID NO: 26), heavy-chain CDR2 comprising the sequence IYPGNVNT (SEQ ID NO: 22), heavy-chain CDR3 comprising the sequence SNPYVMDY (SEQ ID NO: 17), Light-chain CDR1 comprising the sequence QSVNND (SEQ ID NO: 24), Light-chain CDR2 comprising the amino acid sequence YAS (SEQ ID NO: 25), and Light-chain CDR3 comprising the sequence QQAYRSPYT (SEQ ID NO: 20).

According to another aspect, the present invention comprises an antibody-drug conjugate (ADC) comprising a humanized anti-Nectin-4 antibody, or antigen binding portion thereof, conjugated to a toxin selected from the group consisting of MMAE, SN-38, DM1, DM4, and MMAFA, the humanized antibody or antigen binding portion thereof comprising a heavy chain and a light chain, wherein the heavy chain comprises a variable region having an amino acid sequence at least about 90% identical to a sequence selected from the group consisting of SEQ ID NO: 2, SEQ ID NO: 3, SEQ ID NO: 4, SEQ ID NO: 5 and SEQ ID NO: 6; and wherein the light chain comprises a variable region having an amino acid sequence at least about 90% identical to a sequence selected from the group consisting of SEQ ID NO: 8. SEQ ID NO: 9, SEQ ID NO: 10, SEQ ID NO: 11 and SEQ ID NO: 12.

According to some embodiments, the humanized antibody or antigen binding fragment thereof comprises a heavy chain variable region comprising the amino acid sequence QVQL(Q/V)QSG(P/S)ELKKP(E/G)ASVK(I/V)SCKASGYTFTSYYIHWV(K/R)QAPGQG LEW(I/M)GWIYPGNVNTKYNERF(K/Q)GR(A/V)T(L/I)TADKST(N/S)TA(H/Y)MELSSL (T/R)SED(S/T)AVY(F/Y)CARSNPYVMDYWGQGTSVTVSS (SEQ ID NO: 13); and a light chain variable region comprising the amino acid sequence

(SEQ ID NO: 14)
(S/D)I(V/Q)MTQSPS(F/T)L(L/S)AS(A/V)GDRVTITC(K/R)A
SQSVNNDVAWYQQKPG(L/K)AP(E/K)LLMYYASNRFTGVPDRF(T/S)
GSGYGTDFTLTISSLQAED(L/V)A(I/V)YFCQQAYRSPYTFGQGTKL
EIK.

According to some embodiments, the humanized antibody or a fragment thereof comprises a heavy chain and a light chain, wherein the heavy chain comprises a variable region having an amino acid sequence at least about 95% identical to a sequence selected from the group consisting of SEQ ID NO: 2, SEQ ID NO: 3, SEQ ID NO: 4, SEQ ID NO: 5 and SEQ ID NO: 6; and wherein the light chain comprises a variable region having an amino acid sequence at least about 95% identical to a sequence selected from the group consisting of SEQ ID NO: 8, SEQ ID NO: 9, SEQ ID NO: 10, SEQ ID NO: 11 and SEQ ID NO: 12. SEQ ID NOS: 2-6, 8-12 are humanized variants of NTX1105 that were selected based on improved developability and low immunogenicity.

In general, the “NTX1105” includes the chimeric antibody and/or its humanized variants according to the context. The parent chimeric antibody is disclosed in WO 2019/215728 (clone 11). The parent chimeric antibody is named herein NTX1105 (VH0/Vk0).

The humanized variants of NTX1105 include Five heavy chain (VH1 to VH5) and five light chains (Vκ1 to Vκ5). According to some embodiments, the humanized variants comprise the sequence formula set forth in SEQ ID NO: 13 (heavy-chain) and SEQ ID NO: 14 (light-chain).

According to some embodiments, the humanized antibody or a fragment thereof comprises a heavy chain and a light chain, wherein the heavy chain comprises variable region having an amino acid sequence SEQ ID NO: 5 the light chain comprises variable region having an amino acid sequence SEQ ID NO: 12 (denoted herein NTX1105 (VH5/Vk5).

According to some embodiments, the humanized antibody comprises a combination of a heavy chain variable region and a light chain variable region, wherein the combination is selected from the group consisting of: SEQ ID NO: 2 and SEQ ID NO: 8, SEQ ID NO: 2 and SEQ ID NO: 9, SEQ ID NO: 2 and SEQ ID NO: 10, SEQ ID NO: 2 and SEQ ID NO: 11, SEQ ID NO: 2 and SEQ ID NO: 12, SEQ ID NO: 3 and SEQ ID NO: 8, SEQ ID NO: 3 and SEQ ID NO: 9, SEQ ID NO: 3 and SEQ ID NO: 10, SEQ ID NO: 3 and SEQ ID NO: 11. SEQ ID NO: 3 and SEQ ID NO: 12, SEQ ID NO: 4 and SEQ ID NO: 8, SEQ ID NO: 4 and SEQ ID NO: 9. SEQ ID NO: 4 and SEQ ID NO: 10, SEQ ID NO: 4 and SEQ ID NO: 11, SEQ ID NO: 4 and SEQ ID NO: 12, SEQ ID NO: 5 and SEQ ID NO: 8, SEQ ID NO: 5 and SEQ ID NO: 9, SEQ ID NO: 5 and SEQ ID NO: 10, SEQ ID NO: 5 and SEQ ID NO: 11, SEQ ID NO: 5 and SEQ ID NO: 12, SEQ ID NO: 6 and SEQ ID NO: 8, SEQ ID NO: 6 and SEQ ID NO: 9, SEQ ID NO: 6 and SEQ ID NO: 10, SEQ ID NO: 6 and SEQ ID NO: 11, and SEQ ID NO: 6 and SEQ ID NO: 12. Each possibility or a combination of heavy and light chains represents a separate embodiment of the invention.

According to some embodiments, the humanized antibody comprises a combination of a heavy chain variable region and a light chain variable region, wherein the combination is selected from the group consisting of:
i. a heavy chain variable region sequence set forth in SEQ ID NO: 5 and a light chain variable region sequence set forth in SEQ ID NO: 12; and
ii. a heavy chain variable region sequence set forth in SEQ ID NO: 6 and a light chain variable region sequence set forth in SEQ ID NO: 12.

The conjugates of the invention comprise a humanized antibody as described herein. The antibodies include monoclonal antibodies, polyclonal antibodies, multispecific antibodies (for example, bispecific antibodies and polyreactive antibodies), and antibody fragments. Thus, an antibody includes, but is not limited to, full-length, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. The antibody can comprise a human IgG1 constant region. The antibody can comprise a human IgG4 constant region. The antibody can comprise a human kappa light chain.

The term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region. The antibody can comprise a human kappa light chain.

There are several methods known in the art for determining the CDR sequences of a given antibody molecule, but there is no standard unequivocal method. Determination of CDR sequences from antibody heavy and light chain variable regions can be made according to any method known in the art, including but not limited to the methods known as KABAT, Chothia and IMGT. A selected set of CDRs may include sequences identified by more than one method, namely, some CDR sequences may be determined using KABAT and some using IMGT, for example. According to some embodiments, the CDR sequences of the mAb variable regions are determined using the IMGT method. For example, CDR determination is made according to the Kabat (Wu T. T and Kabat E. A., J Exp Med, 1970; 132:211-50) and IMGT (Lefranc M-P, et al., Dev Comp Immunol, 2003, 27:55-77).

When the term “CDR having a sequence”, or a similar term is used, it includes options wherein the CDR comprises the specified sequences and also options wherein the CDR consists of the specified sequence.

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g., scFv or sFv); and multispecific antibodies formed from antibody fragments. In particular embodiments, the antibodies are single-chain antibody fragments comprising a variable heavy chain region and/or a variable light chain region, such as scFvs.

A “humanized” antibody is an antibody in which all or substantially all CDR amino acid residues are derived from non-human CDRs and all or substantially all framework region (FR) amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. According to some embodiments, some FR residues in a humanized antibody are substituted with corresponding residues from a non-human antibody (e.g., the antibody from which the CDR residues are derived), e.g., to restore or improve antibody specificity or affinity.

The amino acid residues in the Fc domain can be substituted to be null, meaning the Fc domain does not bind Fc receptors or can bind with such low affinity and/or avidity as to not cause any Fc receptor signaling as a result of binding. The Fc domain can be null for binding to Fcγ receptors. Some example Fcγ receptors for which the Fc domain can be null for binding can be, but not limited to, FcγRI (CD64), FcγRIIA (CD32a), FcγRIIB (CD32b), FcγRIIIA (CD16a), FcγRIIIA (CD16a) F158 variant, FcγRIIIA (CD16a) V158 variant, or FcγRIIIB (CD16b). The Fc domain may have one or more, two or more, three or more, or four or more amino acid substitutions that decrease binding of the Fc domain to an Fc receptor.

According to some embodiments, the humanized antibody has a mutated Fc domain that prevents FcγR-mediated internalization.

According to some embodiments, the humanized antibody comprises a Fc null domain. According to certain embodiments, the Fc domain is null for binding to a Fcγ receptors.

As used herein, an “Fc null” refers to a domain that exhibits weak to no binding to one or more of the Fcγ receptors.

According to some embodiments, the humanized antibody comprises a Fc null domain. According to certain embodiments, the Fc domain is null for binding to Fcγ receptors found on immune cells. According to certain exemplary embodiments, the Fc domain is null for binding to CD64, CD32a, CD32b, CD16a, and/or CD16b.

According to some embodiments, the humanized antibody comprises a Fc null domain having the LALAPG mutant.

According to some embodiments, the humanized antibody comprising a heavy chain sequence set forth in SEQ ID NO: 51, and a light chain sequence set forth in SEQ ID NO: 52 (NTX1105 (H4/k5) having LALAPG mutant (FcgR^{null})).

The present invention provides conjugates comprising the humanized antibody disclosed herein and a toxin.

According to some embodiments, the toxin is selected from the group consisting of microtubule inhibitor, DNA synthesis inhibitor, topoisomerase inhibitor, and RNA polymerase inhibitor. Each possibility represents a separate embodiment of the invention.

According to certain embodiments, the toxin is a microtubule-destroying drug. According to certain exemplary embodiments, the toxin is auristatin or a derivative thereof. According to certain embodiments, the auristatin derivative is monomethyl auristatin E (MMAE) or monomethyl auristatin F (MMAF). According to some embodiments, the toxin is Exatecan.

According to some embodiments, the toxin is saponin.

According to some embodiments, the toxin is a maytansine derivative. According to certain embodiments, the maytansine derivative is DM4 or DM1.

According to some embodiments, the toxin is quinoline alkaloid. According to certain embodiments, the quinoline alkaloid is SN-38.

According to additional embodiments, the toxin is selected from the group consisting of MMAE, MMAF, Saporin, DM4, DM1, SN-38, Calicheamicin, DXd, PBD, Duocarmycin, Sandramycin, alpha-Amanitin, Chaetocin, CYT997, Daunorubicin, 17-AAG, Agrochelin A, Doxorubicin, Methotrexate, Colchicine, Cordycepin, Epothilone B, Hygrolidin, Herboxidiene, Ferulenol, Curvulin, paclitaxel, Englerin A, Taltobulin, Triptolide, Cryptophycin, and Nemorubicin. Each possibility represents a separate embodiment of the invention.

According to some embodiments, the toxin is SN-38. According to some embodiments, the toxin is DM1. According to some embodiments, the toxin is DM4. According to some embodiments, the toxin is MMAE. According to some embodiments, the toxin is MMAF. According to some embodiments, the toxin is Exatecan.

According to some embodiments, the antibody is directly linked to the toxin. According to other embodiments, the antibody and the toxin are linked through a linker. According to some embodiments, the humanized described herein is covalently linked to the toxin.

According to some embodiments, the linker is cleavable. According to additional embodiments, the linker is not cleavable.

According to some embodiments, the linker is cleaved in response to changes in pH or redox potential. According to some embodiments, the linker is cleaved when contacted with lysosomal enzymes.

According to some embodiments, the linker comprises a portion which is selected from the group consisting of 6-maleimidocaproyl (MC), maleimidopropionyl (MP), valine-citrulline (val-cit), alanine-phenylalanine (ala-phe), ρ-aminobenzyloxycarbonyl (PAB), N-succinimidyl 4-(2-pyridylthio) valerate (SPP), N-succinimidyl 4-(N-maleimidomethyl)-cyclohexane-1-carboxylate (SMCC), N-succinimidyl (4-iodo-acetyl) aminobenzoate (SLAB), 6-maleimidocaproyl-valine-citrulline-ρ-aminobenzyloxycarbonyl (MC-vc-PAB), Val-Cit-PABC, N-succinimidyl-4-(2-pyridyldithio) butanoate (SPDB), N-succinimidyl 3-(pyridin-2-yldithio)-propionate (SPDP), Phe-Lys (Fmoc)-PAB, Aloc-D-Ala-Phe-Lys (Aloc)-PAB-PNP, Boc-Phe-(Alloc) Lys-PAB-PNP, and perfluorophenyl 3-(pyridine-2-yldisulfanyl) propanoate. Each possibility represents a separate embodiment of the invention.

According to some embodiments, the antibody is conjugated to two or more molecules of toxin.

The present invention provides, according to another aspect, a pharmaceutical composition comprising the conjugate described herein and a pharmaceutically acceptable excipient, carrier, or diluent.

According to other embodiments, the pharmaceutical composition according to the invention is for use in treating cancer.

The cancer amendable for treatment by the present invention includes, but is not limited to: carcinoma, lymphoma, blastoma, sarcoma, and leukemia or lymphoid malignancies. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high-grade immunoblastic NHL; high-grade lymphoblastic NHL; high-grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancerous conditions amendable for treatment of the invention include metastatic cancers.

According to other embodiments, the pharmaceutical composition according to the invention are for use in treating cancer characterized by overexpression of Nectin-4. Nectin-4 overexpression related cancer types can be identified using known data bases such as The Cancer Genome Atlas (TCGA). According to certain embodiments, the cancer treatable with a composition according to the present invention is selected from the group consisting of adrenocortical carcinoma (ACC), chromophobe renal cell carcinoma (KICH), liver hepatocellular carcinoma (LIHC), colon and rectal adenocarcinoma (COAD and READ), pancreatic ductal adenocarcinoma (PAAD), pheochromocytoma & paraganglioma (PCPG), papillary kidney carcinoma (KIRP), lung adenocarcinoma (LUAD), head and neck squamous cell carcinoma (HNSC), prostate adenocarcinoma (PRAD), uterine corpus endometrial carcinoma (UCEC), cervical cancer (CESC), cutaneous melanoma (SKCM), mesothelioma (MESO), urothelial bladder cancer (BLCA), clear cell kidney carcinoma (KIRC), lung squamous cell carcinoma (LUSC), uterine carcinosarcoma (UCS), sarcoma (SARC), ovarian serous cystadenocarcinoma (OV), papillary thyroid carcinoma (THCA), glioblastoma multiforme (GBM), breast cancer (BRCA), lower grade glioma (LGG), and diffuse large B-cell lymphoma (DLBC). Each possibility represents a separate embodiment of the invention.

The molecules of the present invention as active ingredients are dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those skilled in the art. In addition, if desired, the composition can contain minor amounts of auxiliary substances such as wetting or emulsifying agents, pH buffering agents.

The pharmaceutical composition according to the present invention may be administered together with an anti-neoplastic composition.

The term “treatment” as used herein refers to both therapeutic treatment and prophylactic or preventative measures. Those in need of treatment include those already with the disorder as well as those in which the disorder is to be prevented. The term “treatment” further relates to alleviation or prevention of symptoms associated with the disease, and/or reducing the severity of the disease.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include melanoma, lung, thyroid, breast, colon, prostate, hepatic, bladder, renal, cervical, pancreatic, leukemia, lymphoma, myeloid, ovarian, uterus, sarcoma, biliary, or endometrial cancer.

According to some embodiments, the method of treating cancer comprises administering the pharmaceutical composition as part of a treatment regimen comprising administration of at least one additional anti-cancer agent.

As used herein the term “individual,” “patient,” or “subject” refers to individuals diagnosed with, suspected of being afflicted with, or at-risk of developing at least one disease for which the described compositions and method are useful for treating. According to some embodiments the individual is a mammal. According to some embodiments, the mammal is a mouse, rat, rabbit, dog, cat, horse, cow, sheep, pig, goat, llama, alpaca, or yak. According to some embodiments, the individual is a human.

As used herein the term an “effective amount” refers to the amount of a therapeutic that causes a biological effect when administered to a mammal. Biological effects include, but are not limited to, reduced tumor growth, reduced tumor metastasis, or prolonged survival of an animal bearing a tumor. A “therapeutic amount” is the concentration of a drug calculated to exert a therapeutic effect. A therapeutic amount encompasses the range of dosages capable of inducing a therapeutic response in a population of individuals. The mammal can be a human individual. The human individual can be afflicted with or suspected or being afflicted with a tumor.

The molecules of the present invention as active ingredients are dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those skilled in the art. In addition, if desired, the composition can contain minor amounts of auxiliary substances such as wetting or emulsifying agents, pH buffering agents.

According to some embodiments, the method of treating cancer comprises administering the pharmaceutical composition as part of a treatment regimen comprising administration of at least one additional anti-cancer agent.

According to some embodiments, the anti-cancer agent is selected from the group consisting of an antimetabolite, a mitotic inhibitor, a taxane, a topoisomerase inhibitor, a topoisomerase II inhibitor, an asparaginase, an alkylating agent, an antitumor antibiotic, and combinations thereof. Each possibility represents a separate embodiment of the invention.

According to some embodiments, the antimetabolite is selected from the group consisting of cytarabine, fludarabine, fluorouracil, mercaptopurine, methotrexate, thioguanine, gemcitabine, and hydroxyurea. According to some embodiments, the mitotic inhibitor is selected from the group consisting of vincristine, vinblastine, and vinorelbine. According to some embodiments, the topoisomerase inhibitor is selected from the group consisting of topotecan and irinotecan. According to some embodiments, the alkylating agent is selected from the group consisting of busulfan, n, carmustine, lomustine, chlorambucil, cyclophosphamide, cisplatin, carboplatin, ifosfamide, mechlorethamine, melphalan, thiotepa, dacarbazine, and procarbazine. According to some embodiments, the antitumor antibiotic is selected from the group consisting of bleomycin, dactinomycin, daunorubicin, doxorubicin, idarubicin, mitomycin, mitoxantrone, and plicamycin. According to some embodiments, the topoisomerase II is selected from the group consisting of etoposide and teniposide. Each possibility represents a separate embodiment of the present invention.

Also described herein is a method of making composition for treating a cancer in an individual afflicted with cancer comprising admixing the humanized antibody or the ADC as described herein and a pharmaceutically acceptable excipient, carrier, or diluent. In certain embodiments, the cancer comprises a solid tumor. In certain embodiments, the cancer is selected from the group consisting of glioblastoma, colon cancer, pancreatic cancer, breast cancer, bladder cancer, kidney cancer, head and neck cancer, ovarian cancer, cervical cancer, prostate cancer, and lung cancer.

### EXAMPLES

Reference is now made to the following examples, which together with the above descriptions, illustrate the invention in a non-limiting fashion.

Generally, the nomenclature used herein, and the laboratory procedures utilized in the present invention include molecular, biochemical, microbiological, immunological, and recombinant DNA techniques. Such techniques are well known in the art. Other general references referring to well-known procedures are provided throughout this document for the convenience of the reader.

### Example 1. High Expression of Nectin-4 mRNA Correlates with Poor Survival Probability of Various Cancer Patients

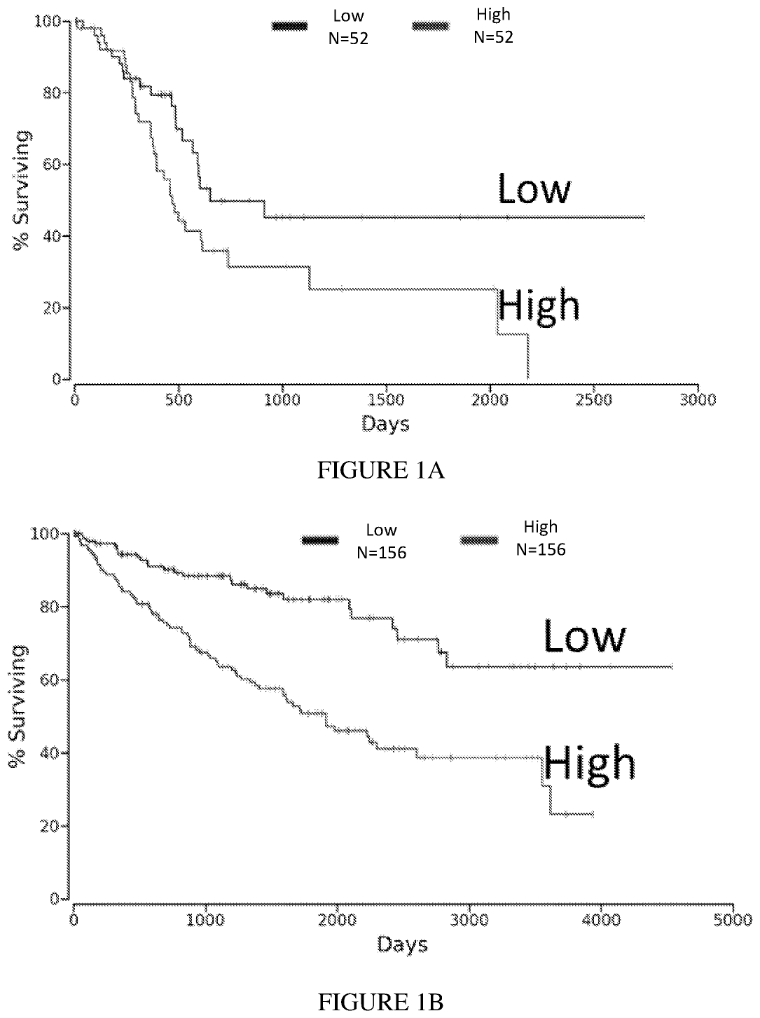

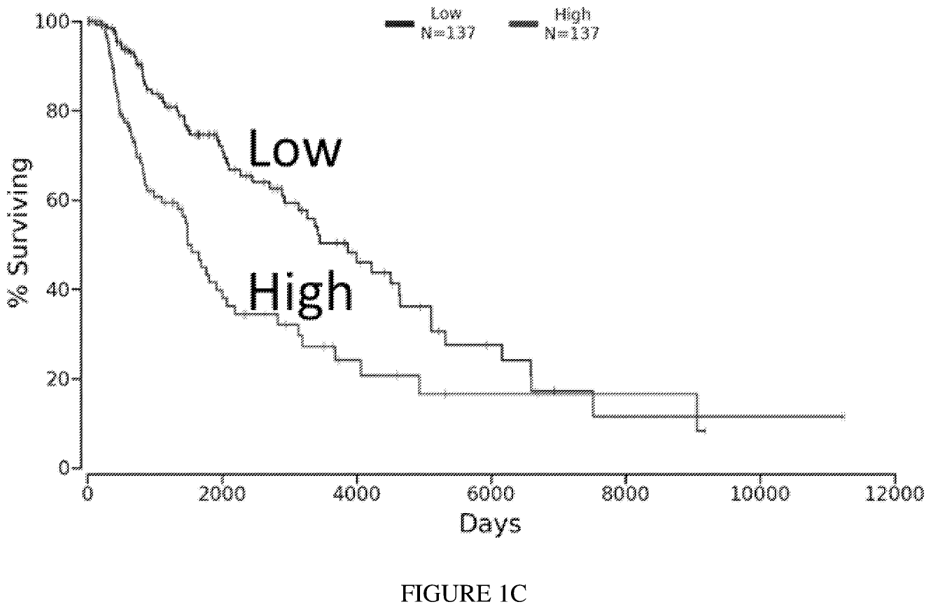

The correlation between Nectin-4 mRNA expression and survival probability was examined on data from TCGA site, analyzed using the oncolnc.org site (https://doi.org/10.7717/peerj-cs.67) and presented in FIGS. 1A-1C. The Nectin-4 mRNA expression levels were the basis for dividing patients for two subgroups of low and high expressors, as indicated by the boxes, the survival advantage of low-expressing patients confirms the detrimental role Nectin-4 plays, and the potential to improve survival by targeting this protein.

### Example 2. Nectin-4 is Expressed at the Protein Level on the Majority of Solid Tumors

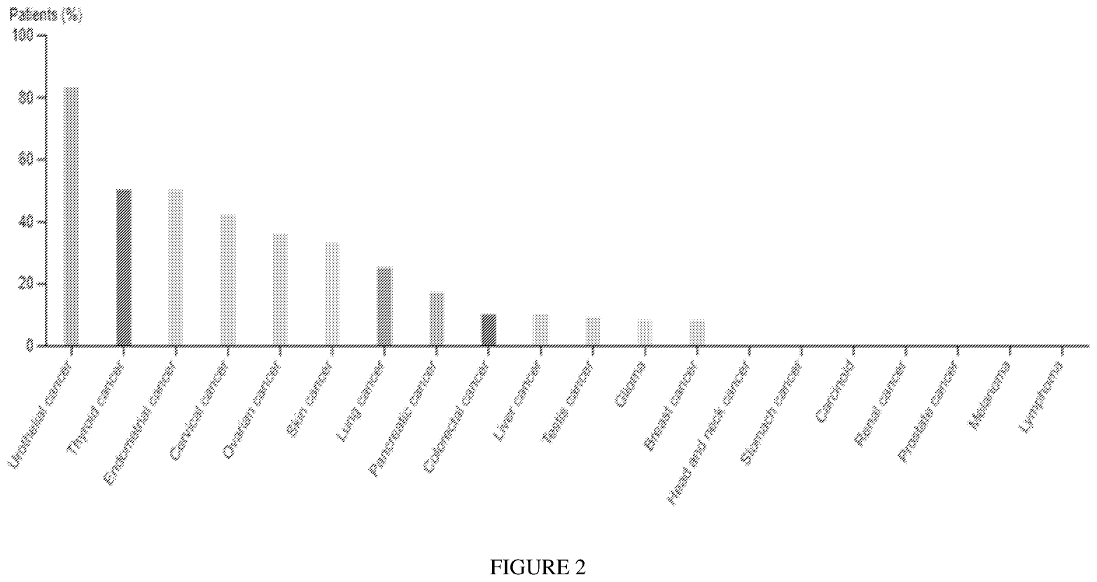

The database Proteinatlas.com was searched for all the aliases of Nectin-4 (NECTIN4, PVRL4). Under the pathology rubric, data using a single mAb was found (HPA010775). The expression data across different tumors is depicted in FIG. 2. The graph is showing percent of tumors positive for Nectin-4 expression. In all indications at least one patient has surface expression of Nectin-4 and in 13/20 indications membranous expression of Nectin-4 is seen at Moderate-High levels.

### Example 3. Various Cytotoxic Agents can be Efficiently Conjugate to Nectin-4 Targeting mAb for the Generation of Nectin-4 ADC

Various linker-payload combinations and the drug-antibody-ratio (DAR), used to conjugate anti-Nectin-4 antibody clone-11 (VH0/Vκ0)-hIgG1 (also described in WO 2019/215728 to the inventors of the present invention) for the production of NTX1105 (VH0/Vκ0) Nectin-4 ADC, are described (Table 1). The cytotoxic agents described in Table 1 include tubulin and TOPO1 targeting agents, and the DAR was selected according to approved ADCs using the mentioned payload.

TABLE 1
Various linker-payload combinations and the drug-antibody-
ratio (DAR), used to conjugate clone-11(VH0/Vκ0)-hIgG1
for the production of NTX1105 (VH0/Vκ0) Nectin-4 ADC.

| | | | Average DAR |
|---|---|---|---|
| Linker | Payload | Release mechanism | (LC-MS) |
| MC-VC-PAB | MMAE | Proteolytic cleavage | 4.2 |
| MC | MMAF | Degradation | 3.8 |
| SMCC | DM1 | Degradation | 3.6 |
| SPDB | DM4 | Redox | 3.5 |
| Lys-PAB-CO | SN38 | pH | 7.5 |

### Example 4. Various Cytotoxic Agents can Induce Tumor Cells Killing when Conjugated to Nectin-4 Targeting mAb In-Vitro

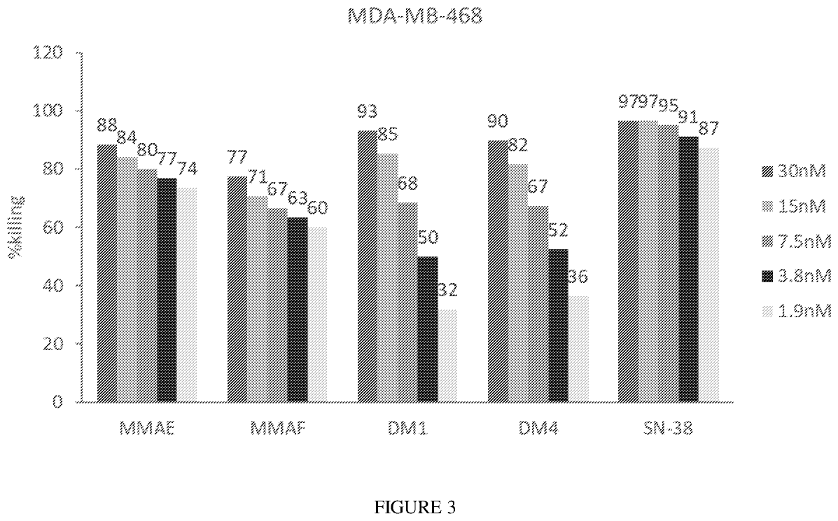

Next, a dose dependent killing activity of the various linker-payload combinations of NTX1105 (VH0/Vκ0) was examined. In vitro assay of TNBC model, MDA-MB-468 cell line was used. The target cells were plated at 2*10³ cells per well and allowed to adhere over 4-6 hours period. The ADCs were added at concentrations of 30-1.9 nM, using 2-fold dilutions, and then the cells were incubated with the ADCs for additional 72 hours. After 72 hours, the assay was harvested and tumor cell killing was evaluated using CellTiter-Glo® 2.0 Cell Viability Assay—(Promega G9242) following standard protocol. The killing data showed robust potency, not reaching EC-50 for most payloads (except DM1/4, which reached EC-50 at ˜3.8 nM), suggesting high potency of the ADC, regardless of the selected linker payload combination (FIG. 3).

### Example 5. Various Cytotoxic Agents can Induce Tumor Cells Killing when Conjugated to Nectin-4 Targeting mAb In-Vivo

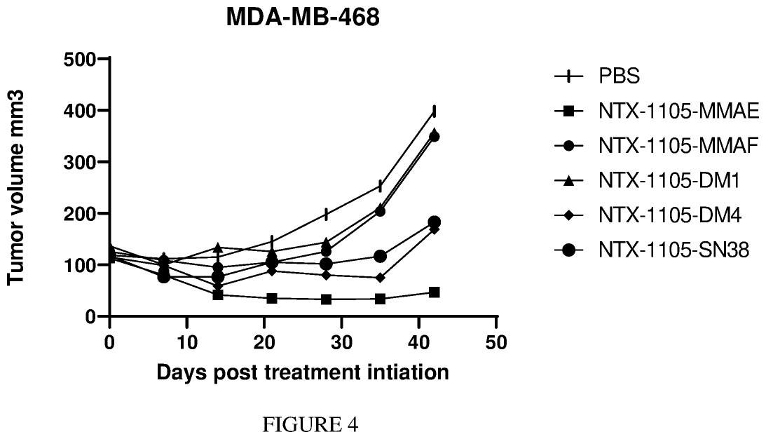

In-vivo efficacy of the of NTX1105 (VH0/Vκ0), anti-Nectin-4 ADCs against the TNBC tumor cells line, MDA-MB-468 (5M cells/mouse) implanted s.c. to 6-week old NOD-SCID female mice, was evaluated. Treatment commenced when tumors reached an average volume of 140 mm³, the animals received 3 doses (Q4D) of 5 mg/kg of NTX1105 with the indicated linker-payloads. FIG. 4 compares the effect of treatment with all NTX1105 combinations of linker payloads (LP). Three of the LP combinations had a significant effect: SN38 and DM4 resulted in significant tumor growth inhibition (TGI, p<0.05), while complete tumor regression was seen for MMAE. The fact that 3 of 5 tested LPs led to significant anti-tumor-activity, suggests the unique utility of NTX1105 as the Ab for the ADC. Based on the results of these experiments, further development of humanized anti-Nectin-4 ADCs based on NTX1105 mAb was done.

### Example 6. Binding of Human Nectin-4 and Cross-Reactivity to Cyno Nectin-4 is Preserved for the Top Selected Humanized Nectin-4 mAbs

In order to assess the binding of all of the variants to human or cynomolgus Nectin-4 antigen and select lead humanized IgGs with the closest affinity to the chimeric (VH0/Vκ0) antibody, single cycle kinetic was performed on supernatants from transfected cell cultures. Kinetic experiments were performed at 25° C. on a Biacore T200 running Biacore T200Control software V2.0.1 and Evaluation software V3.0 (Cytiva, Marlborough, USA). Single cycle kinetic data was obtained using recombinant human (Acro Biosystems, Newark, USA) or cynomolgus Nectin-4 (Acro Biosystems, Newark, USA) as the analyte injected at a flow rate of 40 μl/min to minimize any potential mass transfer effects. A four point, two-fold dilution range from 1.25 nM to 10 nM of antigen in running buffer was used without regeneration between each concentration. The association phases were monitored for 150 seconds for each of the four injections of increasing concentrations of antigen and a single dissociation phase was measured for 250 seconds following the last injection of antigen (in order to improve fit the dissociation was cropped to 175 seconds for human Nectin-4). Regeneration of the sensor chip surface was conducted using a single injection of 3 M MgCl₂. Based on relative KD from Biacore single cycle kinetics analysis on supernatants, six humanized variants (VH4/Vκ1, VH4/Vκ3, VH4/Vκ4, VH4/Vκ5, VH5/Vκ4 and VH5/Vκ5) were selected as lead IgGs for scaled-up production and purification. All selected variants were within the 2.5-fold range of the parental (VH0/Vκ0) clone, suggesting similar activity properties for the humanized variants (Table 2).

TABLE 2
Affinity values of top NTX1105 clones post humanization
relative to the parental mAb (VH0/Vκ0).

| Variant | Human Nectin-4 K_{D} (M) | Cyno Nectin-4 K_{D} (M) |
|---|---|---|
| VH0/Vκ0 | 1.09E−08 | 1.21E−08 |
| VH4/Vκ1 | 1.54E−08 | 1.93E−08 |
| VH4/Vκ3 | 1.55E−08 | 1.94E−08 |
| VH4/Vκ4 | 1.80E−08 | 2.13E−08 |
| VH4/Vκ5 | 1.22E−08 | 1.37E−08 |
| VH5/Vκ4 | 1.77E−08 | 1.92E−08 |
| VH5/Vκ5 | 1.15E−08 | 1.27E−08 |

### Example 7. Improved Characteristics of the Humanized Anti-Nectin-4 mAbs (Reduced Immunogenicity)

The designed VH and Vκ sequences were analyzed for the occurrence of potential T cell epitopes as determined by application of Abzena's proprietary in silico technology, iTope™ (Perry et al. 2008). The iTope™ software predicts favorable interactions between amino acid side chains of a peptide and specific binding pockets (in particular pocket positions; p1, p4, p6, p7 and p9) within the open-ended binding grooves of 34 human MHC class II alleles. These alleles represent the most common HLA-DR alleles found world-wide with no weighting attributed to those found most prevalently in any particular ethnic population. Twenty of the alleles contain the ‘open’ p1 configuration and 14 contain the ‘closed’ configuration where glycine at position 83 is replaced by a valine. The location of key binding residues is achieved by the in-silico generation of 9mer peptides that overlap by eight amino acids spanning the test protein sequence. However, results should be assessed in the light of the fact that all predictive methods for MHC class II binding inherently over-predict the number of T cell epitopes since they do not allow for other important processes during antigen presentation such as protein/peptide processing, recognition by the T cell receptor or T cell tolerance to the peptide. iTope™ analysis was performed with overlapping 9mer peptides (with each overlapping the last peptide by 8 residues). Each 9mer was scored based on the potential ‘fit’ and interactions against each of the 34 MHC class II allotypes with the resultant peptide scores ranging between 0 and 1. MHC Class II epitopes are then defined by iTope™ as follows:
(1) Promiscuous high affinity MHC Class II binding peptides bind>50% of alleles with a majority (17 out of 34 alleles) having a binding score>0.6.
(2) Promiscuous moderate affinity MHC class II binding peptides bind>50% of alleles with a binding score>0.55 (but without a majority>0.6).

These criteria are altered in the case of a large aromatic amino acid (i.e. F, W, Y) occurring in the p1 anchor position where the open p1 pocket of 20 of the 34 alleles allows the binding of a large aromatic residue. Where this occurs, a promiscuous peptide is defined as binding to 10 or more of the subset of 20 alleles. A number of germline promiscuous high and moderate affinity MHC Class II binding ligands were identified in the parental antibody and designed variants however it is unlikely that these epitopes have immunogenic potential due to T cell tolerance, and so were excluded from any further analysis. Overall, the process of humanization resulted in significantly improved mAbs, having lower immunogenicity score, and thus lower associated risk. The least immunogenic variants after this step included heavy chains variants 3-5 and light chain 5 (Table 3).

TABLE 3
Improved characteristics of the humanized anti-Nectin-4 mAbs. Reduced
immunogenicity as measured by predicted MHCII epitopes (less predicted
epitopes correlates to lower immunogenicity) (iTope score) of the
humanized variants as compared to parental. VH0 and humanized heavy
(VH1-5) and light (Vκ1-5) chains used to generate humanized variants
for lead drug selection. Heavy and light chains enabling combinations
resulting in <6 high affinity epitopes are in bold.
Heavy chain:

| Predicted affinity MHCII | VH0 | VH1 | VH2 | VH3 | VH4 | VH5 |
|---|---|---|---|---|---|---|
| Moderate | 7 | 5 | 4 | 4 | 4 | 4 |
| High | 4 | 4 | 4 | 2 | 2 | 1 |

Light chain:

| Predicted affinity MHCII | Vk0 | Vk1 | Vk2 | Vk3 | Vk4 | Vk5 |
|---|---|---|---|---|---|---|
| Moderate | 9 | 4 | 3 | 3 | 3 | 4 |
| High | 9 | 7 | 7 | 6 | 5 | 4 |

### Example 8. Improved Characteristics of the Humanized Anti-Nectin-4 mAbs (Improved Developability)

Thermal ramp stability experiments (Tm and Tagg) are well established methods for ranking proteins and formulations for stability. A protein's denaturation profile provides information about its thermal stability and represents a structural ‘fingerprint’ for assessing structural and formulation buffer modifications. A widely used measure of the thermal structural stability of a protein is the temperature at which it unfolds from the native state to a denatured state.

For many proteins, this unfolding process occurs over a narrow temperature range and the mid-point of this transition is termed ‘melting temperature’ or ‘Tm’. To determine the melting temperature of a protein, UNcle measures the fluorescence of Sypro Orange (which binds to exposed hydrophobic regions of proteins) as the protein undergoes conformational changes. Purified lead antibodies, in duplicate, were diluted to a final test concentration of 0.5 mg/ml in PBS and into which Sypro Orange (at 160× Stock solution) was added to a final concentration of 20× solution. 9 μL of each sample mixture was loaded in duplicate into Uni microcuvettes. Samples were subjected to a thermal ramp from 25-95° C., with a ramp rate of 0.3° C./minute and excitation at 473 nm. Full emission spectra were collected from 250-720 nm, and the area under the curve between 510-680 nm was used to calculate the inflection points of the transition curves (Tonset and Tm). Monitoring of static light scattering (SLS) at 473 nm allowed the detection of protein aggregation, and Tagg (onset of aggregation) was calculated from the resulting SLS profiles. Data analysis was performed using UNcle™ software version 4.0. No significant changes in the TM or Tonset were observed, and significant improvement (>5° C. over VH0/Vκ0) of the stability were seen for two of the humanized variants (VH4/Vκ5 and VH5/Vκ5) (Table 4).

TABLE 4
Characteristics of some of the humanized variants. Depicted are
a summary of thermal stability values for the parental/chimeric
(VH0/Vκ0) antibody and for six purified lead humanized variants
as determined using the UNcle biostability platform. Improved
stability Tagg (measuring of aggregation) by above 5 degrees Celsius,
was considered significant and is marked in bold fonts.

| | Average Tonset | Average Tm1 | (Aggregation) Average |
|---|---|---|---|
| Variant | (° C.) | (° C.) | Tagg (° C.) |
| VH0/Vκ0 | 61.4 | 68.7 | 76.3 |
| VH4/Vκ1 | 61.0 | 68.3 | 74.6 |
| VH4/Vκ3 | 60.6 | 67.9 | 79.6 |
| VH4/Vκ4 | 60.4 | 67.7 | 78.4 |
| VH4/Vκ5 | 59.8 | 67.7 | 81.5 |
| VH5/Vκ4 | 60.5 | 68.2 | 80.1 |
| VH5/Vκ5 | 60.7 | 68.8 | 82 |

### Example 9. Improved Characteristics of the Humanized Anti-Nectin-4 mAbs (Improved Productivity)

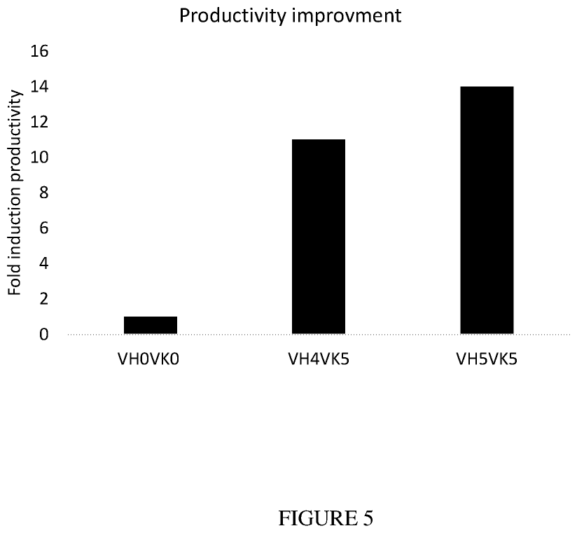

The production of the mAb is a complex process, impacted by the amino acid composition and the structure of the molecule. Transient production is a good indicator of the “productivity” the mAb possess. Chimeric (VH0/Vκ0) and combinations of humanized heavy and light chain DNA constructs were transiently transfected into HEK293 EBNA adherent cells (LGC Standards, Teddington, UK) using a PEI transfection method in 6 well plates and incubated for 6 days post-transfection. Samples were harvested and antibody concentrations were measured on the Octet QK 384 using Protein A biosensors (Molecular Devices, Wokingham, Berkshire, UK), using an IgG1 antibody as standard. The humanization process generally improved the productivity of all mAb (calculated by dividing the titer (mg/ml) of the relevant humanized variant to that of the parental/chimeric VH0/Vκ0), with >10-folds improved productivity of the leading clones (VH4/Vκ5 and VH5/Vκ5) as shown in FIG. 5.

### Example 10. Robust In-Vivo Anti-Tumor Activity of the Lead Humanized mAbs

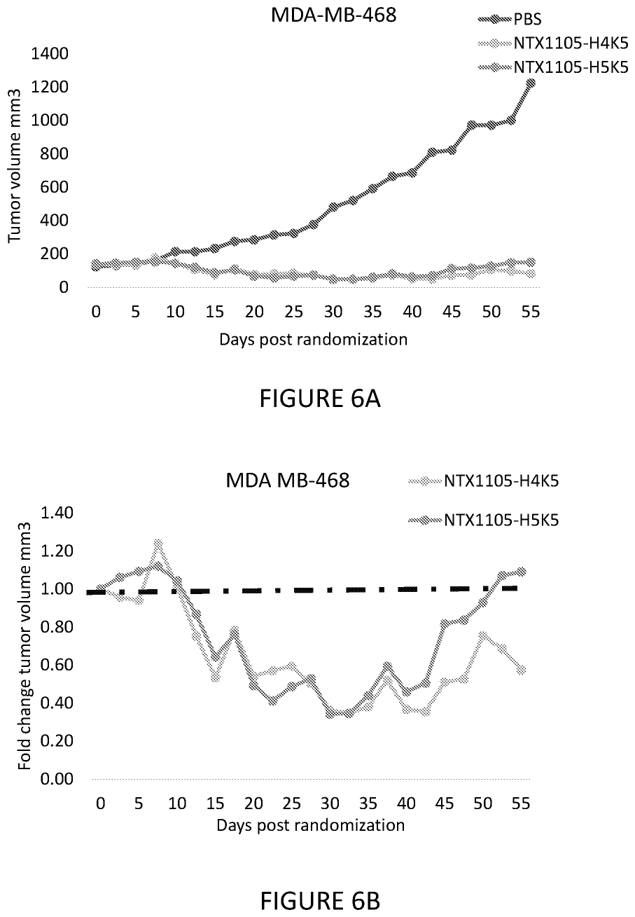

In-vivo efficacy of the of lead humanized NTX1105 (VH4/Vκ5 and VH5/Vκ5), anti-Nectin-4 ADCs against the TNBC tumor cells line, MDA-MB-468 (5M cells/mouse) implanted s.c. to 6 weeks old NOD-SCID female mice, was evaluated. Based on the previous results, MMAE payload was selected. The animals were dosed 4-times (Q4D) with 5 mg/kg of the lead humanized ADCs. As shown in FIG. 6A, both lead ADCs led to tumor regression (above 66% reduction from the treatment start volume). Unexpectedly, clone H4K5, was significantly superior in maintaining the response over time to clone H5K5. The treatment with clone H5K5, after 40 days post last dose resulted in tumor volume similar to treatment initiation (p=0.8) (stasis). In contrast, clone H4K5 even after 40 days post last treatment led to tumor volume significantly lower than the treatment start date (p=0.038) (FIG. 6B). This result is unexpected and suggest superiority of humanized anti-Nectin-4 clone H4K5 over other anti-Nectin-4 humanized mAbs.

### Example 11. Differential Binding and Cell Killing In Vitro Between NTX1105 and Enfortumab-Vedotin In-Vitro

Enfortumab-vedotin (PADCEV), is the only approved ADC targeting Nectin-4. Although having excellent clinical results, PADCEV has severe adverse events, some of which can be attributed to on-target/off-tumor binding, when PADCEVis able to act even at extremely low density of Nectin-4 which is found in some normal tissues. It is here hypothesized, that some of these adverse events can be prevented by an ADC with significantly lower on-target binding/activity.

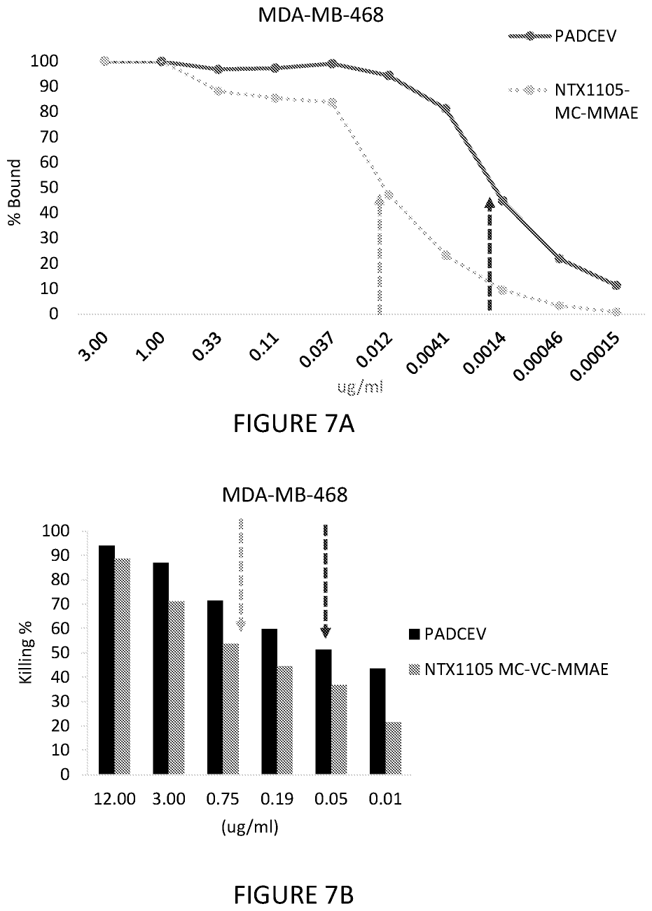

Binding was evaluated on MDA-MB-468 cells. Briefly, 5*10+ cells per well were stained using either PADCEV or NTX1105 conjugated to MMAE at the same DAR as PADCEV. The ADCs were added at concentrations starting 3 μg/ml using 3-fold dilutions. The detection was done using Alexa Fluor® 647 (AffiniPure Fab Fragment Goat Anti-Human IgG (H+L) (cat 109-607-003 Jackson ImmunoResearch). Both ADCs exhibited identical max binding, which is in line with the literature, the EC-50 value of PADCEV was roughly 10 pM (black arrow), roughly 20-folds stronger than NTX1105 (dashed grey arrow) (FIG. 7A). To evaluate the killing capacity of the 2 ADCs in vitro, MDA-MB-468 cells were plated at 2*10³ cells per well and allowed to adhere over 4-6 hours period. The ADCs were added at concentrations of 12-0.01 μg/ml, using 4-fold dilutions, the cells were incubated with the ADCs for additional 72 hours. After 72 hours, the assay was harvested and tumor cell killing was evaluated using CellTiter-Glo® 2.0 Cell Viability Assay-(Promega G9242). The killing data showed roughly 16-folds higher potency in-vitro of PADCEV over NTX1105 (FIG. 7B). Cumulatively, these results, suggest improved safety properties for NTX1105 over PADCEV (the most advanced ADC targeting Nectin-4).

### Example 12. Superior Anti-Tumor Activity In-Vivo of NTX1105 Lead Humanized Clone Compared to Enfortumab-Vedotin (PADCEV)

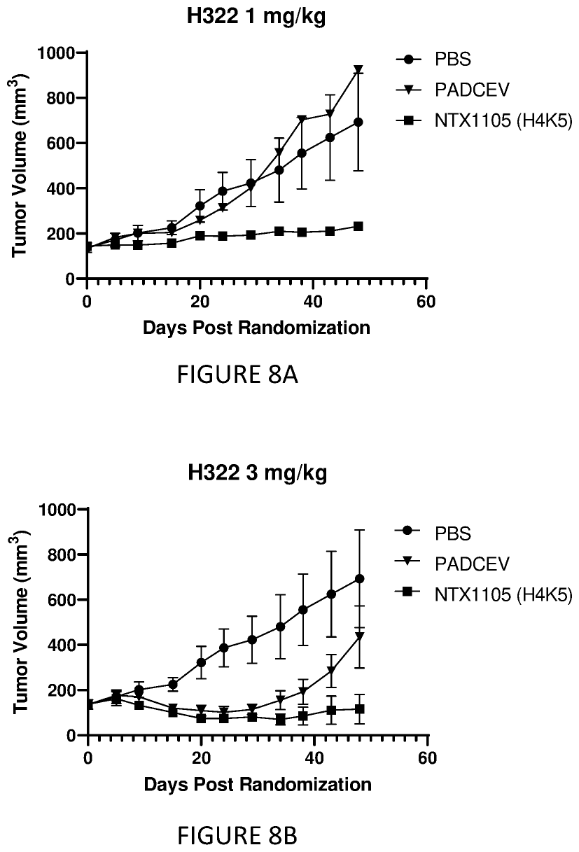

Next, the in-vivo activity of NTX1105 lead humanized clone (VH4/Vκ5) was compared to enfortumab-vedotin (PADCEV). Enfortumab-vedotin is the leading anti-Nectin-4 ADC, already approved for the treatment of patients with urothelial cancer. The lead ADC (humanized NTX1105 (VH4/Vκ5), anti-Nectin-4 ADC) was compared with the PADCEV using an in-vivo efficacy assay against a NSCLC tumor cells line, H322. The tumor cells (5M cells/mouse) were implanted s.c. to 6 weeks old Nude female mice. Treatment commenced when tumors reached 140 mm³, the animals received either 6 doses (Q4D) of PBS (vehicle) or 1 mg/kg of either PADCEV or NTX1105-MC-VC-PAB-MMAE DAR4 (identical conjugation method and DAR to PADCEV), or 3 doses (Q4D) of 3 mg/kg of either PADCEV or NTX1105-MC-VC-PAB-MMAE DAR4 (identical conjugation method and DAR to PADCEV). As shown in FIG. 8A, at the lower dose PADCEV had no effect on tumor growth at any timepoint, at the end of the study, the average TV for PADCEV treated group was slightly above the PBS treated group (924 vs 693 mm³ ns, p=0.48), while NTX1105 was able to stop tumor growth, (220 vs 693 mm³, p=0.04). Moreover, the NTX1105 average TV did not grow significantly from the treatment start date (p=0.28, compared to start treatment date). At the 3 mg/kg dose, both NTX1105 and PADCEV led to tumor regression (46% and 42% reduction of an average TV compared to treatment start by day 20) (FIG. 8B). However, starting day-34 post first dose, NTX1105 ADC was significantly superior to PADCEV in preventing tumor growth during the entire duration of the study. Of note, on day 48 post study start, PADCEV treated group, no longer was significantly superior in terms of tumor volume compared to PBS. NTX1105 ADC treated group led to complete growth arrest of the tumor and had lower tumor volume compared to study start even at day 48 (110-day-48 compared to 138 mm³). These results are unexpected, especially given the significantly stronger in-vitro activity of PADCEV. Moreover, this is the first time in which Nectin-4 targeting ADC (NTX1105) exhibited significant anti-tumor activity in a model in which no biological activity was seen for PADCEV.

### Example 13. Humanized NTX1105 (H4κ5-FcgR^{null}) ADC can Regress H322 Tumors In Vivo with Either Tubulin or TOPO1 Targeting Payloads

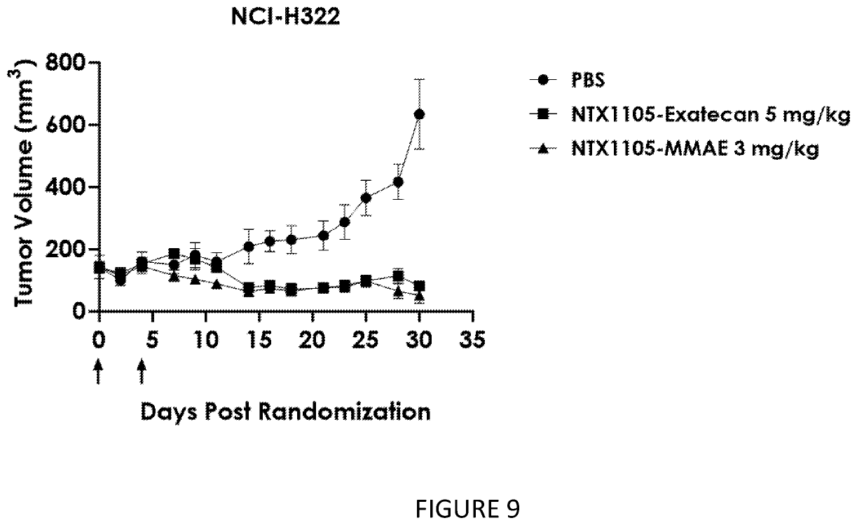

Nude female mice (n=5 per group) were injected SC with 5×10⁶ H322 cells in 1:1 Matrigel. Once tumors reached an average volume of 140 mm³ mice were treated every 4 days, for 2 doses, in a blinded manner, by i.v. injection of either PBS, NTX1105-MMAE-DAR4 at 3 mg/kg or NTX1105-Exatecan-DAR-8 at 5 mg/kg. As shown in FIG. 9, both ADCs were capable of regressing established tumors to the same extent, a surprising outcome considering previous attempts to use TOPO1 targeting payload (FIG. 4). This shows the potential utility of NTX1105 ADC using both MMAE and TOPO1 payloads.

### Example 14. Humanized NTX1105 (H4κ5-FcgR^{null}) ADC with TOPO1 Targeting Payload is Superior to Enfortumab with the Same Payload and DAR

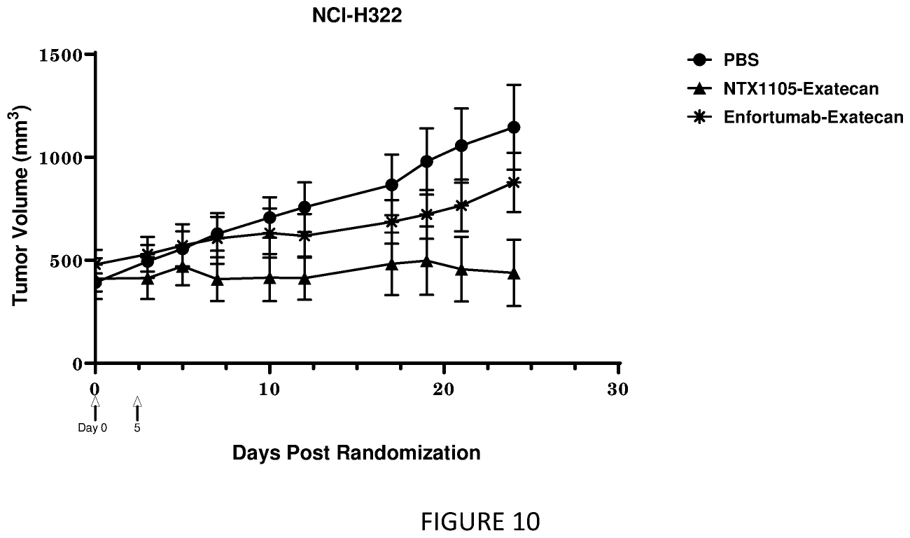

Nude female mice (n=5 per group) were injected SC with 5×10⁶ H322 cells in 1:1 Matrigel. Once tumors reached an average volume of 480 mm³ mice were treated two doses, 5 days apart, in a blinded manner, by i.v. injection of either PBS (vehicle), NTX1105 or Enfortumab based ADCs (both DAR-4 Exatecan) at 10 mg/kg. As shown in FIG. 10, after two 10 mg/kg doses only NTX1105 ADC was able to stop tumor growth, while Enfortumab-Excatecan showed no effect compared to PBS. This shows the potential superiority of NTX1105 ADC over other anti-Nectin-4 based ADCs.

### Sequences

TABLE 5
chimeric antibody and humanized variable regions

| SEQ ID | | |
|---|---|---|
| NO: | Sequence | Chain |
| 1 | QVQLQQSGPELVKPETSVKISCKASGYTFTSYYIHWVKQRPGQGLE | Heavy chain |
| | WIGWIYPGNVNTKYNERFKGKATLTADKSSNTAHMQLTSLTSEDS | (VH0)_chimeric |
| | AVYFCARSNPYVMDYWGQGTSVTVSSASTKGPSVFPLAPSSKSTSGG | |
| | TAALGCLVKDYFPEPVTVSWNSGALTSGVHTFPAVLQSSGLYSLSSVVTV | |
| | PSSSLGTQTYICNVNHKPSNTKVDKRVEPKSCDKTHTCPPCPAPELLGG | |
| | PSVFLFPPKPKDTLMISRTPEVTCVVVDVSHEDPEVKFNWYVDGVEVHN | |
| | AKTKPREEQYNSTYRVVSVLTVLHQDWLNGKEYKCKVSNKALPAPIEKTI | |
| | SKAKGQPREPQVYTLPPSREEMTKNQVSLTCLVKGFYPSDIAVEWESNG | |
| | QPENNYKTTPPVLDSDGSFFLYSKLTVDKSRWQQGNVFSCSVMHEALH | |
| | NHYTQKSLSLSPGK | |
| 2 | QVQLQQSGPELKKPEASVKISCKASGYTFTSYYIHWVKQAPGQGLE | Heavy chain |
| | WIGWIYPGNVNTKYNERFKGRATLTADKSTNTAHMELSSLTSEDSA | VH1 |
| | VYFCARSNPYVMDYWGQGTSVTVSS | |
| 3 | QVQLVQSGSELKKPGASVKISCKASGYTFTSYYIHWVKQAPGQGLE | Heavy chain |
| | WIGWIYPGNVNTKYNERFKGRATLTADKSTNTAYMELSSLRSEDTA | VH2 |
| | VYFCARSNPYVMDYWGQGTSVTVSS | |
| 4 | QVQLVQSGSELKKPGASVKVSCKASGYTFTSYYIHWVRQAPGQGL | Heavy chain |
| | EWIGWIYPGNVNTKYNERFKGRATLTADKSTNTAYMELSSLRSEDT | VH3 |
| | AVYFCARSNPYVMDYWGQGTSVTVSS | |
| 5 | QVQLVQSGSELKKPGASVKVSCKASGYTFTSYYIHWVRQAPGQGL | Heavy chain |
| | EWIGWIYPGNVNTKYNERFKGRVTITADKSTNTAYMELSSLRSEDT | VH4 |
| | AVYYCARSNPYVMDYWGQGTSVTVSS | |
| 6 | QVQLVQSGSELKKPGASVKVSCKASGYTFTSYYIHWVRQAPGQGL | Heavy chain |
| | EWMGWIYPGNVNTKYNERFQGRVTITADKSTSTAYMELSSLRSEDT | VH5 |
| | AVYYCARSNPYVMDYWGQGTSVTVSS | |
| 7 | SIVMTQTPKFLLVSAGDRVTITCKASQSVNNDVAWYQQKPGLSPEL | Light chain |
| | LMYYASNRFTGVPDRFTGSGYGTDFTFTISSVQAEDLAIYFCQQAYR | (Vκ0)_chimeric |
| | SPYTFGGGTKLEIQRTVAAPSVFIFPPSDEQLKSGTASVVCLLNNFYPR | |
| | EAKVQWKVDNALQSGNSQESVTEQDSKDSTYSLSSTLTLSKADYEKHKV | |
| | YACEVTHQGLSSPVTKSFNRGEC | |
| 8 | SIVMTQSPSFLLASAGDRVTITCKASQSVNNDVAWYQQKPGLAPEL | Light chain |
| | LMYYASNRFTGVPDRFTGSGYGTDFTLTISSLQAEDLAIYFCQQAYR | Vκ1 |
| | SPYTFGQGTKLEIK | |
| 9 | SIVMTQSPSTLSASVGDRVTITCKASQSVNNDVAWYQQKPGKAPKL | Light chain |
| | LMYYASNRFTGVPDRFTGSGYGTDFTLTISSLQAEDLAIYFCQQAYR | Vκ2 |
| | SPYTFGQGTKLEIK | |
| 10 | DIQMTQSPSTLSASVGDRVTITCKASQSVNNDVAWYQQKPGKAPKL | Light chain |
| | LMYYASNRFTGVPDRFSGSGYGTDFTLTISSLQAEDLAIYFCQQAYR | Vκ3 |
| | SPYTFGQGTKLEIK | |
| 11 | DIQMTQSPSTLSASVGDRVTITCRASQSVNNDVAWYQQKPGKAPKL | Light chain |
| | LMYYASNRFTGVPDRFSGSGYGTDFTLTISSLQAEDLAIYFCQQAYR | Vκ4 |
| | SPYTFGQGTKLEIK | |
| 12 | DIQMTQSPSTLSASVGDRVTITCRASQSVNNDVAWYQQKPGKAPKL | Light chain |
| | LMYYASNRFTGVPDRFSGSGYGTDFTLTISSLQAEDVAVYFCQQAY | Vκ5 |
| | RSPYTFGQGTKLEIK | |

SEQ ID NO: 13-Heavy chain-General sequence including all humanized variants
QVQL(Q/V)QSG(P/S)ELKKP(E/G)ASVK(I/V)SCKASGYTFTSYYIHWV(K/R)QAPGQG
LEW(I/M)GWIYPGNVNTKYNERF(K/Q)GR(A/V)T(L/I)TADKST(N/S)TA(H/Y)MELSSL
(T/R)SED(S/T)AVY(F/Y)CARSNPYVMDYWGQGTSVTVSS
SEQ ID NO: 14-Light chain-General sequence including all humanized variants
(S/D)I(V/Q)MTQSPS(F/T)L(L/S)AS(A/V)GDRVTITC(K/R)ASQSVNNDVAWYQQKPG
(L/K)AP(E/K)LLMYYASNRFTGVPDRF(T/S)GSGYGTDFTLTISSLQAED(L/V)A(I/V)YF
CQQAYRSPYTFGQGTKLEIK

TABLE 6
CDRs sequences

| Description | KABAT | IMGT | Overlapping sequence |
|---|---|---|---|
| Heavy Chain | SYYIH | GYTFTSYY | SYY |
| CDR1 | SEQ ID NO: 15 | SEQ ID NO: 21 | SEQ ID NO: 26 |
| Heavy Chain | WIYPGNVNTKYNERF(K/Q)G | IYPGNVNT | IYPGNVNT |
| CDR2 | SEQ ID NO: 16 | SEQ ID NO: 22 | SEQ ID NO: 22 |
| | WIYPGNVNTKYNERFKG | | |
| | SEQ ID NO:27 | | |
| | WIYPGNVNTKYNERFQG | | |
| | SEQ ID NO: 28 | | |
| Heavy Chain | SNPYVMDY | ARSNPYVMDY | SNPYVMDY |
| CDR3 | SEQ ID NO: 17 | SEQ ID NO: 23 | SEQ ID NO: 17 |
| Light Chain | (K/R)ASQSVNNDVA | QSVNND | QSVNND |
| CDR1 | SEQ ID NO: 18 | SEQ ID NO: 24 | SEQ ID NO: 24 |
| | KASQSVNNDVA | | |
| | SEQ ID NO: 29 | | |
| | RASQSVNNDVA | | |
| | SEQ ID NO: 30 | | |
| Light Chain | YASNRFT | YAS | YAS |
| CDR2 | SEQ ID NO: 19 | SEQ ID NO: 25 | SEQ ID NO: 25 |
| Light Chain | QQAYRSPYT | QQAYRSPYT | QQAYRSPYT |
| CDR3 | SEQ ID NO: 20 | SEQ ID NO: 20 | SEQ ID NO: 20 |

TABLE 7
Framework (Non-CDR) sequences of the humanized heavy chain variable
regions.

| Chain | FR-H1 | FR-H2 | FR-H3 | FR-H4 |
|---|---|---|---|---|
| VH1 | QVQLQQSGPELKKPEAS | WVKQAPGQGLE | RATLTADKSTNTA | WGQGTSVT |
| | VKISCKASGYTFT | WIG | HMELSSLTSEDSAV | VSS |
| | SEQ ID NO: 31 | SEQ ID NO: 34 | YFCAR | SEQ ID NO: |
| | | | SEQ ID NO: 37 | 41 |
| VH2 | QVQLVQSGSELKKPGA | WVKQAPGQGLE | RATLTADKSTNTA | WGQGTSVT |
| | SVKISCKASGYTFT | WIG | YMELSSLRSEDTAV | VSS |
| | SEQ ID NO: 32 | SEQ ID NO: 34 | YFCAR | SEQ ID NO: |
| | | | SEQ ID NO: 38 | 41 |
| VH3 | QVQLVQSGSELKKPGA | WVRQAPGQGLE | RATLTADKSTNTA | WGQGTSVT |
| | SVKVSCKASGYTFT | WIG | YMELSSLRSEDTAV | VSS |
| | SEQ ID NO: 33 | SEQ ID NO: 35 | YFCAR | SEQ ID NO: |
| | | | SEQ ID NO: 38 | 41 |
| VH4 | QVQLVQSGSELKKPGA | WVRQAPGQGLE | RVTITADKSTNTAY | WGQGTSVT |
| | SVKVSCKASGYTFT | WIG | MELSSLRSEDTAVY | VSS |
| | SEQ ID NO: 33 | SEQ ID NO: 35 | YCAR | SEQ ID NO: |
| | | | SEQ ID NO: 39 | 41 |
| VH5 | QVQLVQSGSELKKPGA | WVRQAPGQGLE | RVTITADKSTSTAY | WGQGTSVT |
| | SVKVSCKASGYTFT | WMG | MELSSLRSEDTAVY | VSS |
| | SEQ ID NO: 33 | SEQ ID NO: 36 | YCAR | SEQ ID NO: |
| | | | SEQ ID NO: 40 | 41 |

TABLE 8
Framework (Non-CDR) sequences of the humanized light variable regions.

| Chain | FR-L1 | FR-L2 | FR-L3 | FR-L4 |
|---|---|---|---|---|
| LK1 | SIVMTQSPSFLLASA | WYQQKPGLAPELL | GVPDRFTGSGYGTD | FGQGTKLE |
| | GDRVTITC | MY | FTLTISSLQAEDLAI | IK |
| | SEQ ID NO: 42 | SEQ ID NO: 45 | YFC | SEQ ID NO: |
| | | | SEQ ID NO: 47 | 50 |
| LK2 | SIVMTQSPSTLSASV | WYQQKPGKAPKLL | GVPDRFTGSGYGTD | FGQGTKLE |
| | GDRVTITC | MY | FTLTISSLQAEDLAI | IK |
| | SEQ ID NO: 43 | SEQ ID NO: 46 | YFC | SEQ ID NO: |
| | | | SEQ ID NO: 47 | 50 |
| LK3 | DIQMTQSPSTLSASV | WYQQKPGKAPKLL | GVPDRFSGSGYGTD | FGQGTKLE |
| | GDRVTITC | MY | FTLTISSLQAEDLAI | IK |
| | SEQ ID NO: 44 | SEQ ID NO: 46 | YFC | SEQ ID NO: |
| | | | SEQ ID NO: 48 | 50 |
| LK4 | DIQMTQSPSTLSASV | WYQQKPGKAPKLL | GVPDRFSGSGYGTD | FGQGTKLE |
| | GDRVTITC | MY | FTLTISSLQAEDLAI | IK |
| | SEQ ID NO: 44 | SEQ ID NO: 46 | YFC | SEQ ID NO: |
| | | | SEQ ID NO: 48 | 50 |
| LK5 | DIQMTQSPSTLSASV | WYQQKPGKAPKLL | GVPDRFSGSGYGTD | FGQGTKLE |
| | GDRVTITC | MY | FTLTISSLQAEDVA | IK |
| | SEQ ID NO: 44 | SEQ ID NO: 46 | VYFC | SEQ ID NO: |
| | | | SEQ ID NO: 49 | 50 |

Full sequence of NTX1105 (H4/k5) having LALAPG mutant (FcgR^{null})-Heavy chain-
SEQ ID NO: 51:
QVQLVQSGSELKKPGASVKVSCKASGYTFTSYYIHWVRQAPGQGLEWIGWIYPGNV
NTKYNERFKGRVTITADKSTNTAYMELSSLRSEDTAVYYCARSNPYVMDYWGQGT
SVTVSS
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAP
EAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALGAPIEKTISKAKGQPR
EPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
Full sequence of NTX1105 (H4/k5)-light chain-SEQ ID NO: 52
DIQMTQSPSTLSASVGDRVTITCRASQSVNNDVAWYQQKPGKAPKLLMYYASNRFT
GVPDRFSGSGYGTDFTLTISSLQAEDVAVYFCQQAYRSPYTFGQGTKLEIKRTVAAPS
VFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC

1-31. (canceled)

32. A humanized antibody that specifically binds human Nectin-4, or a fragment thereof comprising at least the antigen binding site, wherein the antibody or the fragment thereof comprises a heavy chain and a light chain, wherein the heavy chain comprises a variable region having an amino acid sequence at least about 90% identical to a sequence selected from the group consisting of SEQ ID NO: 2, SEQ ID NO: 3, SEQ ID NO: 4, SEQ ID NO: 5 and SEQ ID NO: 6; and wherein the light chain comprises a variable region having an amino acid sequence at least about 90% identical to a sequence selected from the group consisting of SEQ ID NO: 8, SEQ ID NO: 9, SEQ ID NO: 10, SEQ ID NO: 11 and SEQ ID NO: 12, wherein the humanized antibody or a fragment thereof comprises a set of six CDR sequences, wherein the heavy-chain CDR1 comprising the sequence SYY (SEQ ID NO: 26), heavy-chain CDR2 comprising the sequence IYPGNVNT (SEQ ID NO: 22), heavy-chain CDR3 comprising the sequence SNPYVMDY (SEQ ID NO: 17), Light-chain CDR1 comprising the sequence QSVNND (SEQ ID NO: 24), Light-chain CDR2 comprising the amino acid sequence YAS (SEQ ID NO: 25), and Light-chain CDR3 comprising the sequence QQAYRSPYT (SEQ ID NO: 20).

33. The humanized antibody of claim 32, wherein the humanized antibody or a fragment thereof comprises a heavy chain and a light chain, wherein the heavy-chain variable region comprises the sequence set forth in SEQ ID NO: 13, and the light-chain variable region comprises the sequence set forth in SEQ ID NO: 14.

34. The humanized antibody of claim 32, wherein the humanized antibody or a fragment thereof comprises a set of six CDR sequences, wherein heavy-chain CDR1 comprising the sequence SYYIH (SEQ ID NO: 15), heavy-chain CDR2 comprising the sequence WIYPGNVNTKYNERF(K/Q)G (SEQ ID NO: 16), heavy-chain CDR3 comprising the sequence SNPYVMDY (SEQ ID NO: 17), Light-chain CDR1 comprising the sequence (K/R)ASQSVNNDVA (SEQ ID NO: 18), Light-chain CDR2 comprising the sequence YASNRFT (SEQ ID NO: 19), and Light-chain CDR3 comprising the sequence QQAYRSPYT (SEQ ID NO: 20).

35. The humanized antibody of claim 32, wherein the humanized antibody or antigen binding fragment thereof comprising: a heavy chain variable region and a light chain variable region, the heavy chain variable region comprising a set of four framework (FR) sequences: (A) FR-H1 selected from the group consisting of SEQ ID NOs: 31, 32, and 33; (B) FR-H2 selected from the group consisting of SEQ ID NOs: 34, 35, and 36; (C) FR-H3 selected from the group consisting of SEQ ID NOs: 37, 38, 39, and 40; (D) FR-H4 is SEQ ID NO: 41; and the light chain variable region comprising a set of four framework (FR) sequences: (A) FR-L1 selected from the group consisting of SEQ ID NOs: 42, 43, and 44; (B) FR-L2 selected from the group consisting of SEQ ID NOs: 45 and 46; (C) FR-L3 selected from the group consisting of SEQ ID NOs: 47, 48 and 49; and (D) FR-L4 is SEQ ID NO: 50.

36. The humanized antibody of claim 32, wherein the heavy chain variable region of the humanized monoclonal antibody comprises a sequence selected from the group consisting of SEQ ID NOs: 2, 3, 4, 5, and 6; and the light chain variable region of the humanized monoclonal antibody comprises a sequence selected from the group consisting of SEQ ID NOs: 8, 9, 10, 11, and 12.

37. The humanized antibody of claim 32, wherein the heavy chain variable region of the humanized monoclonal antibody comprises SEQ ID NO: 5, and the light chain variable region of the humanized monoclonal antibody comprises SEQ ID NO: 12.

38. A conjugate comprising the humanized antibody of claim 32.

39. The conjugate of claim 38, comprising a cytotoxic moiety, a radioactive moiety, or a labeling tag.

40. A pharmaceutical composition comprising the humanized antibody or antigen binding fragment according to claim 32, or a conjugate comprising said antibody and a pharmaceutically acceptable excipient, carrier, or diluent.

41. A method of treating a cancer in an individual in need of such treatment, the method comprising administering to the individual a therapeutically effective amount of a pharmaceutical composition according to claim 40.

42. An antibody-drug conjugate (ADC) comprising a humanized anti-Nectin-4 antibody, or antigen binding portion thereof, conjugated to a toxin, wherein the antibody or antigen binding portion thereof comprises a set of six CDR sequences, wherein heavy-chain CDR1 comprising the sequence SYYIH (SEQ ID NO: 15), heavy-chain CDR2 comprising the sequence WIYPGNVNTKYNERF(K/Q)G (SEQ ID NO: 16), heavy-chain CDR3 comprising the sequence SNPYVMDY (SEQ ID NO: 17), Light-chain CDR1 comprising the sequence (K/R)ASQSVNNDVA (SEQ ID NO: 18), Light-chain CDR2 comprising the sequence YASNRFT (SEQ ID NO: 19), and Light-chain CDR3 comprising the sequence QQAYRSPYT (SEQ ID NO: 20).

43. The antibody-drug conjugate of claim 42, the antibody or a fragment thereof comprises a heavy chain and a light chain, wherein the heavy chain comprises a variable region having an amino acid sequence at least about 90% identical to a sequence selected from the group consisting of SEQ ID NO: 2, SEQ ID NO: 3, SEQ ID NO: 4, SEQ ID NO: 5 and SEQ ID NO: 6; and wherein the light chain comprises a variable region having an amino acid sequence at least about 90% identical to a sequence selected from the group consisting of SEQ ID NO: 8, SEQ ID NO: 9, SEQ ID NO: 10, SEQ ID NO: 11 and SEQ ID NO: 12.

44. The antibody-drug conjugate of claim 42, wherein the heavy chain variable region of the humanized antibody comprises an amino acid sequence set forth in SEQ ID NO: 13; and the light chain variable region of the humanized antibody comprises an amino acid sequence set forth in SEQ ID NO: 14.

45. The antibody-drug conjugate of claim 42, wherein the heavy chain variable region of the humanized antibody comprises an amino acid sequence set forth in SEQ ID NO: 5; and the light chain variable region of the humanized antibody comprises an amino acid sequence set forth in SEQ ID NO: 12.

46. The antibody-drug conjugate of claim 42, wherein the toxin is selected from the group consisting of microtubule inhibitor, DNA synthesis inhibitor, topoisomerase inhibitor and RNA polymerase inhibitor.

47. The antibody-drug conjugate of claim 46, wherein the toxin is selected from the group consisting of Exatecan, monomethyl auristatin E (MMAE), monomethyl auristatin F (MMAF), saporin, DM4, DM1, and SN-38.

48. The antibody-drug conjugate of claim 42, wherein the antibody and the toxin are linked through a linker.

49. A pharmaceutical composition comprising the antibody-drug conjugate according to claim 42 and a pharmaceutically acceptable excipient, carrier, or diluent.

50. A method of treating a cancer in an individual in need of such treatment, the method comprising administering to the individual a therapeutically effective amount of a pharmaceutical composition according to claim 49.

51. The method of claim 50, wherein the method of treating cancer comprises administering or performing at least one additional anti-cancer therapy.
